(19) United States
(12) Patent Application Publication
(10) Pub. No.: US 2009/0305894 A1
(43) Pub. Date: Dec. 10, 2009

(54) use of sulfonanilides as herbicide

(76) Inventors: Koichi Araki, Ibaraki (JP); Yoshitaka Sato, Ibaraki (JP); Takuya Gomibuchi, Ibaraki (JP); Keiji Endo, Ibaraki (JP); Shinichi Shirakura, Tochigi (JP); Shin Nakamura, Saitama (JP); Christopher Rosinger, Hofheim (DE); Dieter Feucht, Eschborn (DE)

Correspondence Address: FROMMER LAWRENCE & HAUG, 745 FIFTH AVENUE- 10TH FL., NEW YORK, NY, 10151, US

(21) Appl. No.: 12/066,940

(22) PCT Filed: Sep. 2, 2006
(86) PCT No.: PCT/EP06/08591

§ 371 Date: Aug. 5, 2009

(30) Foreign Application Priority Data
Sep. 16, 2005 (JP) 2005-269562
Jul. 4, 2006 (JP) 2006-184101

(51) Int. Cl. A01N 43/66; C07D 251/30; C07D 239/52; A01N 43/54; A01P 13/00

(52) U.S. Cl. 504/227; 544/219; 544/319; 504/243

(57) ABSTRACT

Herbicidal compositions comprising containing sulfonanilides of the formula (I), wherein R¹ represents hydrogen, fluorine, chlorine, C₁₋₄ alkyl, C₁₋₄ alkoxy, C₃₋₆ cycloalkyl-C₁₋₄ alkyloxy or C₁₋₄ haloalkoxy, R² represents hydrogen, fluorine or chlorine, R³ represents hydrogen or fluorine, R⁴ represents hydrogen or C₁₋₄ alkyl which may be optionally C₁₋₄ alkoxy-substituted, C₃₋₆ alkenyl or C₃₋₆ alkynyl, R⁵ represents hydrogen, R⁶ represents hydroxy, fluorine or chlorine, or R⁵ and R⁶ may form, together with the carbon to which they are bonded, C═O, and X represents CH or N, provided that the following cases are excluded: (i) R¹ represents hydrogen, fluorine or chlorine, R² represents hydrogen, R³ represents hydrogen, R⁴ represents hydrogen, R⁵ represents hydrogen, and R⁶ represents hydroxy, (ii) R¹ represents hydrogen, fluorine or chlorine, R² represents hydrogen, R³ represents hydrogen, R⁴ represents hydrogen, and R⁵ and R⁶ form C═O together with the carbon to which they are bonded, (iii) R¹ represents C₁₋₄ alkyl, R² represents hydrogen, R³ represents hydrogen, R⁴ represents hydrogen, R⁵ represents hydrogen, R⁶ represents hydroxy, and X represents CH, or (iv) R¹ represents C₁₋₄ alkyl, R² represents hydrogen, R³ represents hydrogen, R⁴ represents hydrogen, R⁵ and R⁶ form C═O together with the carbon to which they are bonded, and X represents CH, and new compounds being embraced by the formula (I).

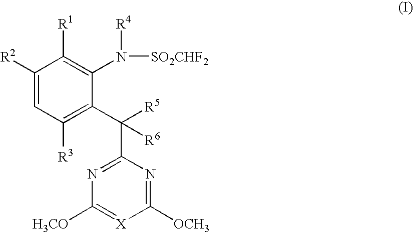

The present invention relates to a use of sulfonanilides as herbicides, to novel sulfonanilides, to a process for their preparation, and to novel intermediates.

It has been known that some kinds of sulfonanilides are effective as herbicides (e.g., WO93/9099 and WO96/41799, Japanese Patent Application Laid-Open (KOKAI) Nos. 11-60562 and 2000-44546, and Japanese Patent Application Laid-Open No. 2006-56870) and also it has been known that some of sulfonanilides are effective as fungicide (e.g., Japanese Patent Application Laid-Open No. 2006-56871).

There have now been found that sulfonanilides of the formula (I) show excellent herbicidal activities;

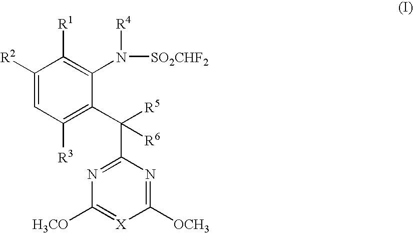

wherein

R¹ represents hydrogen, fluorine, chlorine, C₁₋₄ alkyl, C₁₋₄ alkoxy, C₃₋₆ cycloalkyl-C₁₋₄ alkyloxy or C₁₋₄ haloalkoxy,

R² represents hydrogen, fluorine or chlorine,

R³ represents hydrogen or fluorine,

R⁴ represents hydrogen or C₁₋₄ alkyl which may be optionally C₁₋₄ alkoxy-substituted, C₃₋₆ alkenyl or C₃₋₆ alkynyl,

R⁵ represents hydrogen,

R⁶ represents hydroxy, fluorine or chlorine, or

R⁵ and R⁶ may form, together with the carbon to which they are bonded, C═O, and

X represents CH or N,

provided that the following cases are excluded:

(i) R¹ represents hydrogen, fluorine or chlorine, R² represents hydrogen, R³ represents hydrogen, R⁴ represents hydrogen, R⁵ represents hydrogen, and R⁶ represents hydroxy,

(ii) R¹ represents hydrogen, fluorine or chlorine, R² represents hydrogen, R³ represents hydrogen, R⁴ represents hydrogen, and R⁵ and R⁶ form C═O together with the carbon to which they are bonded,

(iii) R¹ represents C₁₋₄ alkyl, R² represents hydrogen, R³ represents hydrogen, R⁴ represents hydrogen, R⁵ represents hydrogen, R⁶ represents hydroxy, and X represents CH, or

(iv) R¹ represents C₁₋₄ alkyl, R² represents hydrogen, R³ represents hydrogen, R⁴ represents hydrogen, R⁵ and R⁶ form C═O together with the carbon to which they are bonded, and X represents CH.

The sulfonanilides of the above formula (I) include known compounds described in Japanese Patent Application Laid-Open (KOKAI) No. 2006-56871.

The following sulfonanilides of the formulae (IA), (IB) and (IC) being embraced by the aforementioned formula (I), according to the present invention, are novel compounds;

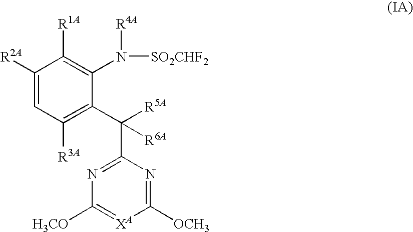

wherein

R^{1A} represents methyl, ethyl, methoxy, ethoxy, n-propyloxy, isopropyloxy, n-butyloxy, isobutyloxy, cyclopropylmethyloxy or difluoromethoxy,

R^{2A} represents hydrogen, fluorine or chlorine,

R^{3A} represents hydrogen or fluorine,

R^{4A} represents hydrogen, methyl, ethyl, n-propyl, n-butyl, methoxymethyl, ethoxymethyl, allyl, 2-butenyl, propargyl or 2-butynyl,

R^{5A} represents hydrogen,

R^{6A} represents hydroxy, or

R^{5A} and R^{6A} may form, together with the carbon to which they are bonded, C═O, and

X^{A} represents CH or N,

provided that the following cases are excluded:

(i) R^{1A} represents methyl or ethyl, R^{2A} represents hydrogen, R^{3A} represents hydrogen, R^{4A} represents hydrogen, R^{5A} represents hydrogen, R^{6A} represents hydroxy, and X^{A} represents CH,

(ii) R^{1A} represents methyl or ethyl, R^{2A} represents hydrogen, R^{3A} represents hydrogen, R^{4A} represents hydrogen, and R^{5A} and R^{6A} form C═O together with the carbon to which they are bonded, and X^{A} represents CH,

(iii) R^{1A} represents methoxy or difluoromethoxy, R^{2A} represents hydrogen, R^{3A} represents hydrogen, R^{4A} represents hydrogen, R^{5A} represents hydrogen, R^{6A} represents hydroxy, or R^{5A} and R^{6A} form, together with the carbon to which they are bonded, C═O, and X^{A} represents CH, or

(iv) R^{1A} represents methyl, R^{2A} represents fluorine, R^{3A} represents hydrogen, R^{4A} represents hydrogen, R^{5A} represents hydrogen, R^{6A} represents hydroxy, and X^{A} represents CH,

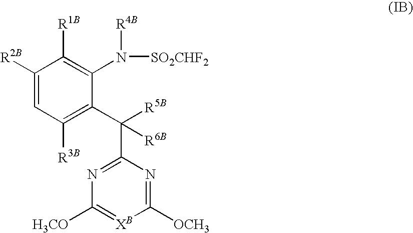

wherein

R^{1B} represents fluorine or chlorine,

R^{2B} represents hydrogen,

R^{3B} represents hydrogen,

R^{4B} represents ethyl, n-propyl, n-butyl, methoxymethyl, allyl, 2-butenyl, propargyl or 2-butynyl,

R^{5B} represents hydrogen,

R^{6B} represents hydroxy, or

R^{5B} and R^{6B} may form, together with the carbon to which they are bonded, C═O, and

X^{B} represents N,

and

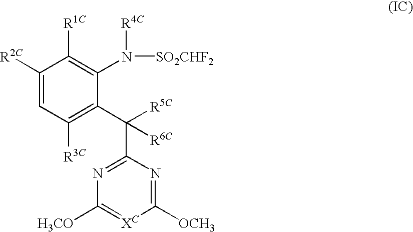

wherein

R^{1C} represents fluorine,

R^{2C} represents fluorine,

R^{3C} represents hydrogen,

R^{4C} represents hydrogen,

R^{5C} represents hydrogen,

R^{6C} represents hydroxy, fluorine or chlorine, and

X^{C} represents CH or N,

provided that

(i) where X^{C} represents N, then R^{6C} represents hydroxy, or

(ii) where X^{C} represents CH, then R^{6C} represents fluorine or chlorine.

The compounds of the formulae (IA), (IB) and (IC) have not been described in any known literatures.

The compounds of the formula (IA) can be obtained by a process in which

(a) Preparation of the compounds of the formula (IA) wherein R^{4A} represents hydrogen and R^{5A} and R^{6A} form C═O, together with the carbon to which they are bonded:

compounds of the formula (II)

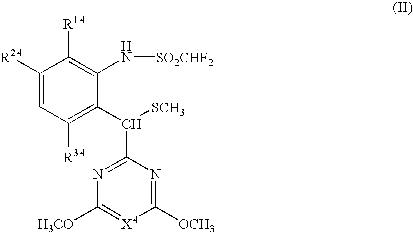

wherein R^{1A}, R^{2A}, R^{3A} and X^{A} have the same definition as aforementioned,

are reacted with hydrogen peroxide and acetic acid in the presence of inert solvents,
or
(b) Preparation of the compounds of the formula (IA) wherein R^{4A} represents hydrogen and R^{5A} and R^{6A} form C═O, together with the carbon to which they are bonded:

compounds of the formula (III)

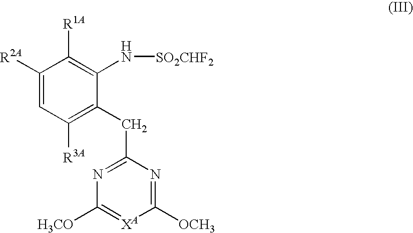

wherein R^{1A}, R^{2A}, R^{3A} and X^{A} have the same definition as aforementioned,

are reacted an oxidizing agent in the presence of inert solvents, and if appropriate, in the presence of an acid catalyst,
or
(c) Preparation of the compounds of the formula (IA) wherein R^{4A} represents hydrogen, R^{5A} represents hydrogen and R^{6A} represents hydroxy:

compounds of the formula (IAc)

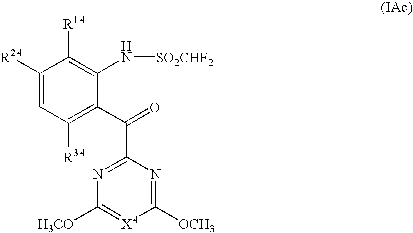

wherein R^{1A}A, R^{2A}, R^{3A} and X^{A} have the same definition as aforementioned,

are reacted with an alkali metal hydride complex or a borane complex, in the presence of inert solvents,
or
(d) Preparation of the compounds of the formula (IA) wherein R^{4A} represents methyl, ethyl, n-propyl, n-butyl, methoxymethyl, ethoxymethyl, allyl, 2-butenyl, propargyl or 2-butynyl:

compounds of the formula (IAd)

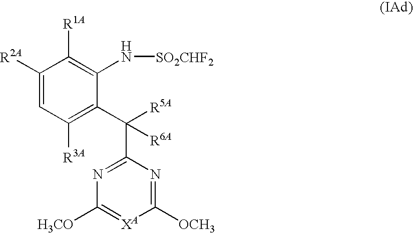

wherein R^{1A}, R^{2A}, R^{3A}, R^{5A}, R^{6A} and X^{A} have the same definition as aforementioned,

are reacted with compounds of the formula (IV)

R^{4Ad}−L^{d} (IV)

wherein R^{4Ad} represents methyl, ethyl, n-propyl, n-butyl, methoxymethyl, ethoxymethyl, allyl, 2-butenyl, propargyl or 2-butynyl, and L^{d} represents halogen,

in the presence of inert solvents, and if appropriate, in the presence of an acid binder.

The compounds of the formula (IB) can be obtained by a process in which

(e) compounds of the formula (V)

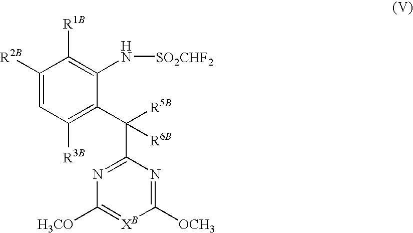

wherein R^{1B}, R^{2B}, R^{3B}, R^{5B}, R^{6B} and X^{B} have the same definition as aforementioned,

are reacted with compounds of the formula (VI)

R^{4Be}−L^{e} (VI)

wherein R^{4Be} represents ethyl, n-propyl, n-butyl, methoxymethyl, ethoxymethyl, allyl, 2-butenyl, propargyl or 2-butynyl, and L^{e} represents halogen,

in the presence of inert solvents, and if appropriate, in the presence of an acid binder.

The compounds of the formula (IC) can be obtained by a process in which

(f) Preparation of a compound of the formula (IC) wherein R^{4C} represents hydrogen, R^{5C} represents hydrogen, R^{6C} represents hydroxy and X^{C} represents N:

a compound of the formula (VII)

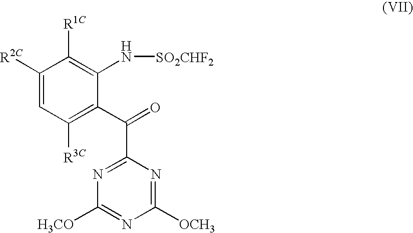

wherein R^{1C}, R^{2C} and R^{3C} have the same definition as aforementioned, are reacted with an alkali metal hydride complex or a borane complex, in the presence of inert solvents,

or
(g) Preparation of a compound of the formula (IC) wherein R^{4C} represents hydrogen, R^{5C} represents hydrogen, R^{6C} represents fluorine or chlorine and X^{C} represents CH:

a compound of the formula (ICg)

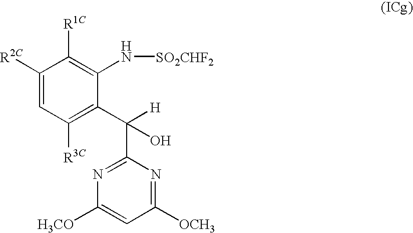

wherein R^{1C}, R^{2C} and R^{3C} have the same definition as aforementioned, are reacted with a halogenating agent, in the presence of inert solvents.

The compounds of the formula (I) including the novel compounds of the formulae (IA), (IB) and (IC) show strong herbicidal activity.

The sulfonanilides of the formula (I) are generically embraced by the general formulae described in WO93/9099 or WO96/41799 mentioned above, however the compounds of the present formula (I) are not specifically disclosed in WO93/9099 or WO96/41799. The sulfonanilides of the present formula (I) are also generically embraced by the general formula described in Japanese Patent Application Laid-Open No. 2006-56871, and a part of them are described in Japanese Patent Application Laid-Open No. 2006-56871. Unexpectedly, the compounds of the formula (I) show practically remarkably outstanding herbicidal activity as compared with the known compounds having analogous structures and specifically described in WO93/9099 and WO96/41799, and also show an excellent herbicidal effect on the sulfonylurea resistant weeds, together with excellent selectivity between crops and weeds.

In the present specification,

“C₁₋₄ Alkyl” can be straight-chain or branched-chain and there can be mentioned, for example, methyl, ethyl, n- or iso-propyl, n-, iso-, sec- or tert-butyl, etc.

“C₃₋₆ Alkenyl” can be straight-chain or branched-chain and there can be mentioned, for example, allyl, 2-butenyl, 3-butenyl, etc.

“C₃₋₆ Alkynyl” can be straight-chain or branched-chain and there can be mentioned, for example, propargyl (2-propynyl), 2-butynyl, 3-butynyl, etc.

“C₁₋₄ Alkoxy” can be straight-chain or branched-chain and there can be mentioned, for example, methoxy, ethoxy, n- or iso-propyloxy, n-, iso-, sec- or tert-butoxy, etc.

As “C₃₋₆ cycloalkyl-C₁₋₄ Alkyloxy” there can be mentioned, for example, cyclopropylmethyloxy, etc.

“C₁₋₄ haloalkoxy” represents alkoxy whose hydrogen is substituted with halogen and there can be mentioned, for example, difluoromethoxy, trifluoromethoxy, 2-fluoroethoxy, 2-chloroethoxy, 2-bromoethoxy, 2,2,2-trifluoroethoxy, 2,2,2-trichloroethoxy, 3-chloropropoxy, etc.

As “C₁₋₄ Alkyl which is substituted with C₁₋₄ alkoxy” in “C₁₋₄ alkyl which may be optionally substituted with C₁₋₄ alkoxy”, in which alkoxy part can be of the same definition as the aforementioned “alkoxy” and alkyl part can be of the same definition as the aforementioned “alkyl”, there can be mentioned, for example, methoxymethyl, ethoxymethyl, etc.

In the compounds of the formula (I) according to the invention, there can be mentioned, as a preferable group of compounds, the compounds in which

R¹ represents hydrogen, fluorine, chlorine, methyl, ethyl, methoxy, ethoxy, n-propyloxy, isopropyloxy, n-butyloxy, isobutyloxy, cyclopropylmethyloxy or difluoromethoxy,

R² represents hydrogen, fluorine or chlorine,

R³ represents hydrogen or fluorine,

R⁴ represents hydrogen, methyl, ethyl, n-propyl, n-butyl, methoxymethyl, ethoxymethyl, allyl, 2-butenyl, propargyl or 2-butynyl,

R⁵ represents hydrogen,

R⁶ represents hydroxy, fluorine or chlorine, or

R⁵ and R⁶ may form, together with the carbon to which they are bonded, C═O, and

X represents CH or N,

provided that the following cases are excluded:

(i) R¹ represents hydrogen, fluorine or chlorine, R² represents hydrogen, R³ represents hydrogen, R⁴ represents hydrogen, R⁵ represents hydrogen, and R⁶ represents hydroxy,

(ii) R¹ represents hydrogen, fluorine or chlorine, R² represents hydrogen, R³ represents hydrogen, R⁴ represents hydrogen, and R⁵ and R⁶ form C═O together with the carbon to which they are bonded,

(iii) R¹ represents methyl or ethyl, R² represents hydrogen, R³ represents hydrogen, R⁴ represents hydrogen, R⁵ represents hydrogen, R⁶ represents hydroxy, and X represents CH, or

(iv) R¹ represents methyl or ethyl, R² represents hydrogen, R³ represents hydrogen, R⁴ represents hydrogen, R⁵ and R⁶ form C═O together with the carbon to which they are bonded, and X represents CH.

In the compounds of the formula (I) according to the invention, there can be mentioned, as a more preferable group of compounds, the compounds in which

R¹ represents fluorine, chlorine, methyl, ethyl or methoxy,

R² represents hydrogen or fluorine,

R³ represents hydrogen,

R⁴ represents hydrogen, methyl, ethyl, n-propyl, n-butyl, methoxymethyl, ethoxymethyl, allyl, 2-butenyl, propargyl or 2-butynyl,

R⁵ represents hydrogen,

R⁶ represents hydroxy, or

R⁵ and R⁶ may form, together with the carbon to which they are bonded, C═O, and

X represents N,

provided that the following cases are excluded:

(i) R¹ represents fluorine or chlorine, R² represents hydrogen, R³ represents hydrogen, R⁴ represents hydrogen, R⁵ represents hydrogen, and R⁶ represents hydroxy, or

(ii) R¹ represents fluorine or chlorine, R² represents hydrogen, R³ represents hydrogen, R⁴ represents hydrogen, and R⁵ and R⁶ form C═O together with the carbon to which they are bonded.

The aforementioned compounds show an excellent effect as herbicides for, e.g., directly seeded paddy rice and/or transplanted paddy-rice.

In addition, in the compounds of the formula (I) according to the invention, there can also be mentioned, as a more preferable group of compounds, the compounds in which

R¹ represents hydrogen, fluorine, chlorine, methyl, methoxy, ethoxy, n-propyloxy, isopropyloxy, n-butyloxy, isobutyloxy, cyclopropylmethyloxy or difluoromethoxy,

R² represents hydrogen, fluorine or chlorine,

R³ represents hydrogen or fluorine,

R⁴ represents hydrogen, methyl, ethyl, n-propyl, n-butyl, methoxymethyl, ethoxymethyl, allyl, 2-butenyl, propargyl or 2-butynyl,

R⁵ represents hydrogen,

R⁶ represents hydroxy, fluorine or chlorine, and

X represents CH,

provided that the following cases are excluded:

(i) R¹ represents hydrogen, fluorine or chlorine, R² represents hydrogen, R³ represents hydrogen, R⁴ represents hydrogen, R⁵ represents hydrogen, and R⁶ represents hydroxy, or

(ii) R¹ represents methyl, R² represents hydrogen, R³ represents hydrogen, R⁴ represents hydrogen, R⁵ represents hydrogen, R⁶ represents hydroxy, and X represents CH.

The aforementioned compounds show an excellent effect as herbicides for, e.g., directly seeded paddy rice, transplanted paddy-rice and/or Poaceae field crops (e.g., wheat).

The compounds of the formula (I) can include geometrical isomers and rotational isomers.

The preparation method (a) can be represented by the following reaction scheme in the case of using as the starting materials, for example, 3-fluoro-2-methoxy-6-[(4,6-dimethoxypyrimidin-2-yl)(methylthio)methyl]-N-difluor omethanesulfonanilide, aqueous hydrogen peroxide and acetic acid.

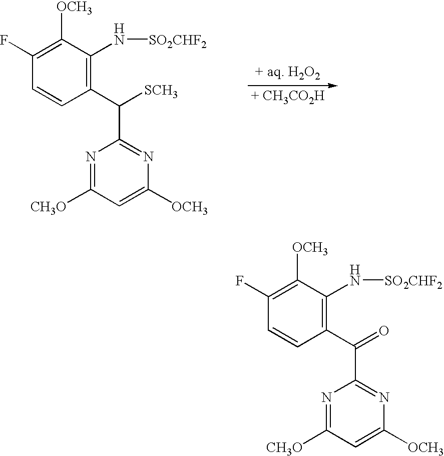

The preparation method (b) can be represented by the following reaction scheme in the case of using as the starting materials, for example, 2-methoxy-6-[(4,6-dimethoxytriazin-2-yl)methyl]-N-difluoromethanesulfonanilide and chromium (VI) oxide as a oxdizing agent.

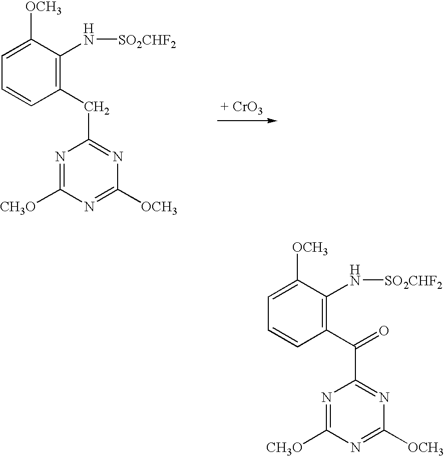

The preparation method (c) can be represented by the following reaction scheme in the case of using as the starting materials, for example, 2-methoxy-6-[(4,6-dimethoxytriazin-2-yl)carbonyl]-N-difluoromethanesulfonanilide and sodium borohydride as a reducing agent.

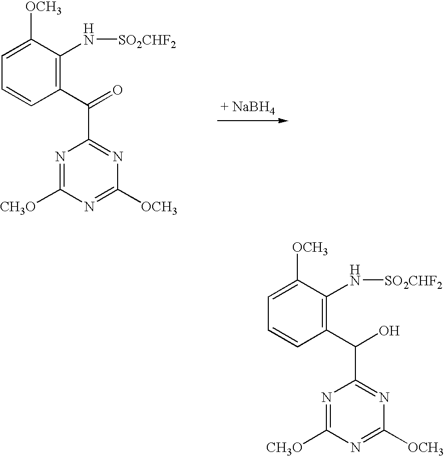

The preparation method (d) can be represented by the following reaction scheme in the case of using as the starting materials, for example, 2-methoxy-6-[(4,6-dimethoxytriazin-2-yl)carbonyl]-N-difluoromethanesulfonanilide, methyl iodide and potassium carbonate as an acid binding agent.

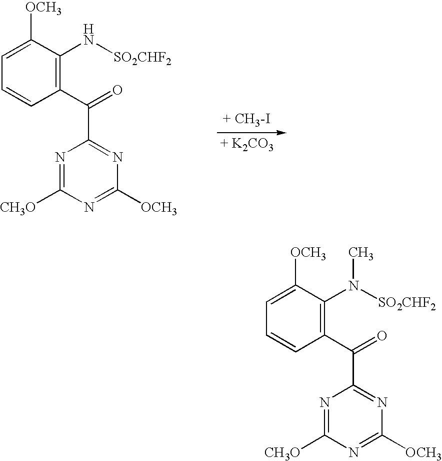

The preparation method (e) can be represented by the following reaction scheme in the case of using as the starting materials, for example, 2-fluoro-6-[(4,6-dimethoxypyrimidin-2-yl)carbonyl]-N-difluoromethanesulfonanilide, allyl bromide and potassium carbonate as an acid binding agent.

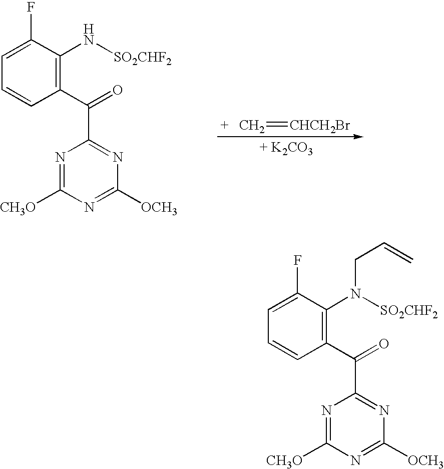

The preparation method (f) can be represented by the following reaction scheme in the case of using as the starting materials, for example, 2,3-difluoro-6-[(4,6-dimethoxypyrimidin-2-yl)carbonyl]-N-difluoromethanesulfonanilide and sodium borohydride as a reducing agent.

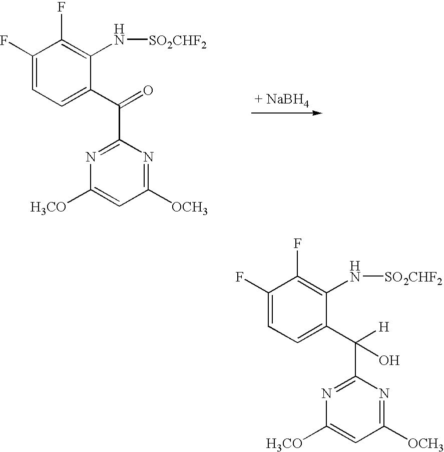

The preparation method (g) can be represented by the following reaction scheme in the case of using as the starting materials, for example, 2,3-difluoro-6-[1-(4,6-dimethoxypyrimidin-2-yl)-1-hydroxymethyl]-N-difluoromethanesulfonanilide and diethylaminesulfur trifluoride as a halogenating agent.

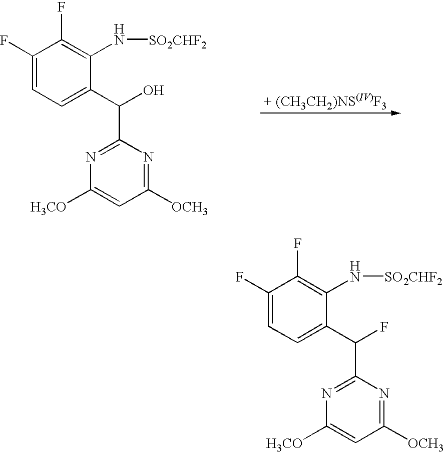

The compounds of the formula (II) used as the starting materials in the preparation method (a) are novel compounds, and can be prepared by reacting, for example, compounds of the formula (VIII)

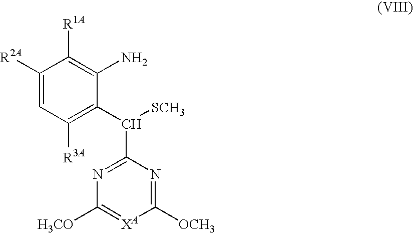

wherein R^{1A}, R^{2A}, R^{3A} and X^{A} have the same definition as aforementioned,

with difluoromethanesulfonyl chloride in accordance with the method described in Japanese Patent Application Laid-Open Nos. 2006-56870 or 2006-56871.

The compounds of the formula (VIII) are novel compounds, and can be prepared by reacting, for example, compounds of the formula (IX)

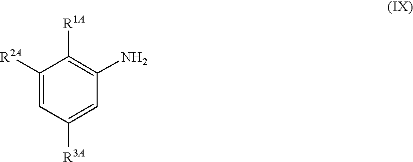

wherein R^{1A}, R^{2A} and R^{3A} have the same definition as aforementioned, with 2-methylthiomethyl-4,5-dimethoxypyrimidine or 2-methylthiomethyl-4,6-dimethoxytriazine under the presence of tert-butyl hypochlorite in accordance with the method described, for example, in WO96/41799.

Difluoromethanesulfonyl chloride, the compounds of the formula (IX), 2-methylthiomethyl-4,5-dimethoxypyrimidine and 2-methylthiomethyl-4,6-dimethoxytriazine are conventionally known compounds per se.

As specific examples of the compounds of the formula (II), there can be mentioned as follows:
2-methoxy-6-[1-(4,6-dimethoxypyrimidin-2-yl)-1-methylthiomethyl]-N-difluoromethanesulfonanilide,
3-fluoro-2-methyl-6-[1-(4,6-dimethoxypyrimidin-2-yl)-1-methylthiomethyl]-N-difluoromethanesulfonanilide,
3-fluoro-2-methoxy-6-[1-(4,6-dimethoxypyrimidin-2-yl)-1-methylthiomethyl]-N-difluoromethanesulfonanilide,
2-difluoromethoxy-6-[1-(4,6-dimethoxypyrimidin-2-yl)-1-methylthiomethyl]-N-difluoromethanesulfonanilide and so on.

As specific examples of the compounds of the formula (VIII), there can be mentioned as follows:
2-methoxy-6-[1-(4,6-dimethoxypyrimidin-2-yl)-1-methylthiomethyl]aniline,
3-fluoro-2-methyl-6-[1-(4,6-dimethoxypyrimidin-2-yl)-1-methylthiomethyl]aniline,
3-fluoro-2-methoxy-6-[1-(4,6-dimethoxypyrimidin-2-yl)-1-methylthiomethyl]aniline,
2-difluoromethoxy-6-[1-(4,6-dimethoxypyrimidin-2-yl)-1-methylthiomethyl]aniline and so on.

The compounds of the formula (III) used as the starting materials in the preparation method (b) are novel compounds, and can be prepared by reacting, for example, compounds of the formula (X)

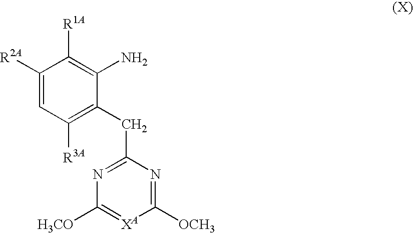

wherein R^{1A}, R^{2A}, R^{3A} and X^{A} have the same definition as aforementioned,

with difluoromethanesulfonyl chloride in accordance with the method described, for example, in Japanese Patent Application Laid-Open Nos. 2006-56870 or 2006-56871.

The compounds of the above-mentioned formula (X) are novel compounds, and can be prepared by reducing, for example, the compounds of the afore-mentioned formula (VIII) in accordance with the method described in WO96/41799 or Japanese Patent Application Laid-Open No. 2006-56871.

As specific examples of the compounds of the formula (III), there can be mentioned as follows:
2-methyl-6-[(4,6-dimethoxytriazin-2-yl)-methyl]-N-difluoromethanesulfonanilide,
2-methoxy-6-[(4,6-dimethoxytriazin-2-yl)methyl]-N-difluoromethanesulfonanilide,
2-ethyl-6-[(4,6-dimethoxytriazin-2-yl)-methyl]-N-difluoromethanesulfonanilide,
2-methyl-3-fluoro-6-[(4,6-dimethoxytriazin-2-yl)-methyl]-N-difluoromethanesulfonanilide and so on.

As specific examples of the compounds of the formula (X), there can be mentioned as follows:
2-methyl-6-[(4,6-dimethoxytriazin-2-yl)-methyl]aniline,
2-methoxy-6-[(4,6-dimethoxytriazin-2-yl)-methyl]aniline,
2-ethyl-6-[(4,6-dimethoxytriazin-2-yl)-methyl]aniline,
2-methyl-3-fluoro-6-[(4,6-dimethoxytriazin-2-yl)-methyl]aniline and so on.

As the reducing agents reacted with the compounds of the above-mentioned formula (VIII), there can be mentioned, for example, combination of sodium borohydride and nickel (II) chloride, or Raney nickel, etc.

As the oxidizing agents used in the preparation method (b), there can be mentioned, for example, chromium (VI) oxide, manganese dioxide, selenium dioxide, etc.

The compounds of the formula (IAc), used as the starting materials in the aforementioned preparation process (c), correspond to a part of the compounds of the formula (IA) of the present invention, that can be prepared by the aforementioned preparation process (a) or (b), and as their specific examples there can be mentioned as follows:
2-methoxy-6-[(4,6-dimethoxypyrimidin-2-yl)carbonyl]-N-difluoromethanesulfonanilide,
3-fluoro-2-methyl-6-[(4,6-dimethoxypyrimidin-2-yl)carbonyl]-N-difluoromethanesulfonanilide,
3-fluoro-2-methoxy-6-[(4,6-dimethoxypyrimidin-2-yl)carbonyl]-N-difluoromethanesulfonanilide,
2-methyl-6-[(4,6-dimethoxytriazin-2-yl)carbonyl]-N-difluoromethanesulfonanilide,
2-methoxy-6-[(4,6-dimethoxytriazin-2-yl)carbonyl]-N-difluoromethanesulfonanilide,
2-ethyl-6-[(4,6-dimethoxytriazin-2-yl)carbonyl]-N-difluoromethanesulfonanilide and so on.

As the alkaline metal hydride complex compound or borane complex used in the aforementioned preparation process (c) there can be mentioned, for example, sodium borohydride, lithium aluminium hydride, dimethyl sulfide borane, pyridine-borane and so on.

The compounds of the formula (IAd), used as the starting materials in the aforementioned preparation process (d), correspond to a part of the compounds of the formula (IA) of the present invention that can be prepared by the aforementioned preparation process (a), (b) or (c), and as their specific examples, there can be mentioned as follows:
2-methyl-6-[(4,6-dimethoxytriazin-2-yl)carbonyl]-N-difluoromethanesulfonanilide,
2-methoxy-6-[(4,6-dimethoxytriazin-2-yl)carbonyl]-N-difluoromethanesulfonanilide,
2-methoxy-6-[(4,6-dimethoxypyrimidin-2-yl)carbonyl]-N-difluoromethanesulfonanilide,
2-methyl-6-[1-(4,6-dimethoxytriazin-2-yl)-1-hydroxymethyl]-N-difluoromethanesulfonanilide,
2-methoxy-6-[1-(4,6-dimethoxytriazin-2-yl)-1-hydroxymethyl]-N-difluoromethanesulfonanilide and so on.

The compounds of the formula (V) used as the starting materials in the aforementioned preparation process (e), are known per se, and can be prepared in accordance with the method described in Japanese Patent Application Laid-Open Nos. 2006-56870, and there can be mentioned, for example, 2-fluoro-6-[(4,6-dimethoxytriazin-2-yl)-carbonyl]-N-difluoromethanesulfonanilide, 2-chloro-6-[(4,6-dimethoxytriazin-2-yl)carbonyl]-N-difluoromethanesulfonanilide, 2-fluoro-6-[1-(4,6-dimethoxytriazin-2-yl)-1-hydroxymethyl]-N-difluoromethanesulfonanilide, 2-chloro-6-[1-(4,6-dimethoxytriazin-2-yl)-1-hydroxymethyl]-N-difluoromethanesulfonanilide and so on.

The compounds of the formula (IV) in the preparation process (d) and the compounds of the formula (VI) in the preparation process (e) are known per se, and, specifically, there can be mentioned as follows:

methyl iodide, ethyl iodide, n-propyl iodide, n-butyl iodide, chloromethyl methyl ether, chloromethyl ethyl ether, allyl bromide, propargyl bromide and so on.

The compounds of the formula (VII) used as the starting materials in the aforementioned preparation process (f) are novel compounds, and can be prepared by oxdizing, for example, compounds of the formula (XI)

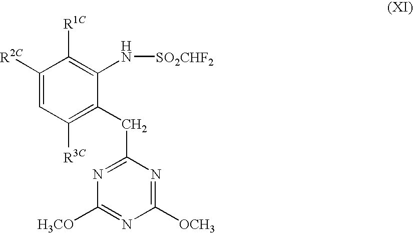

wherein R^{1C}, R^{2C} and R^{3C} have the same definition as aforementioned, in accordance with the aforementioned preparation process (b).

The compounds of the aforementioned formula (XI) are novel compounds, and can be prepared by reacting, for example, 2,3-difluoro-6-[(4,6-dimethoxytriazin-2-yl)methyl]aniline, which is known per se, with difluoromethanesulfonyl chloride in accordance with the method described in Japanese Patent Application Laid-Open Nos. 2006-56870 or 2006-56871.

As specific examples of the compounds of the formula (VII), there can be mentioned, for example, 2,3-difluoro-6-[(4,6-dimethoxytriazin-2-yl)carbonyl]-N-difluoromethansulfonanilide and so on.

As the alkali metal hydride complex compound or borane complex used in the preparation process (f), there can be mentioned the same as in the above process (c).

The compounds of the formula (ICg) used in the preparation method (g) are encompassed by a part of the compounds of the formula (I) of the invention, or are known compounds per se described in Japanese Patent Application Laid-Open No. 2006-56870 or 2006-56871 mentioned above. The compound of the formula (ICg) can be prepared by reducing a compound of the formula (XII)

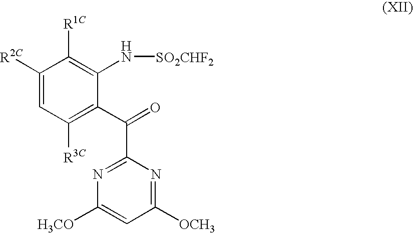

wherein R^{1C}, R^{2C}, and R^{3C} have the same definition as aforementioned, in accordance with the preparation method (c) mentioned above.

Specific example of the compound of the formula (ICg) is 2,3-difluoro-6-[1-(4,6-dimethoxypyrimidin-2-yl)-1-hydroxymethyl]-N-difluorometha nesulfonanilide and so on.

The compound of the formula (XII) is conventionally a known compound per se and can be prepared in accordance with the method described in Japanese Patent Application Laid-Open No. 2006-56870 or 2006-56871 mentioned above. The compound of the formula (XII) can also be prepared by allowing a known compound per se of the following formula (XIII)

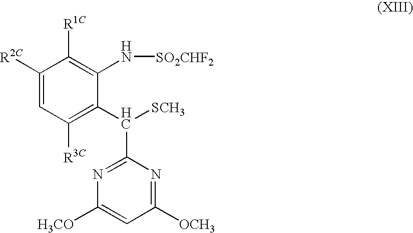

wherein R^{1C}, R^{2C}, and R^{3C} have the same definition as aforementioned, to react in hydrogen peroxide and acetic acid in accordance with a known reaction in the field of organic chemistry, so-called Pummerer rearrangement reaction, as set forth in Reference Example 4.

The halogenating agents used in the preparation method (g) are known per se, which include diethylaminesulfur trifluoride, phosphorus oxychloride, and thionyl chloride.

The compounds of the formula (IA) wherein R^{4A} represents methyl, ethyl, n-propyl, n-butyl, methoxymethyl, ethoxymethyl, allyl, 2-butenyl, propargyl or 2-butynyl can alternatively be prepared by reacting the 1 mol of the compounds of the formula (II) with about 2 to about 5 moles of the compounds of the formula (IV) in the presence of about 2 to about 5 moles of acid binding agents as shown in Reference Example 2 hereinafter.

The compounds of the formulae (II), (III), (VIII) and (X), as either starting materials or intermediate products, are novel compounds and have not been described in the literature.

Those compounds can be represented collectively by the following two formulae (XIV) and (XV):
Compounds of the formula (XIV)

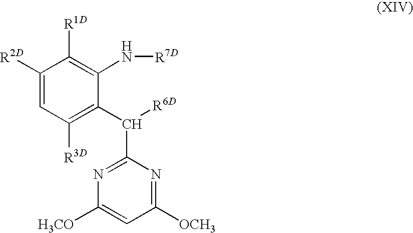

wherein

R^{1D} represents methyl, methoxy, ethoxy, n-propyloxy, isopropyloxy, n-butyloxy, isobutyloxy, cyclopropylmethyloxy or difluoromethoxy,

R^{2D} represents hydrogen, fluorine or chlorine,

R^{3D} represents hydrogen or fluorine,

R^{6D} represents hydrogen or methylthio, and

R^{7D} represents hydrogen or difluoromethanesulfonyl,

provided that the following cases are excluded:

(i) R^{1D} represents methoxy or difluoromethoxy, R^{2D} represents hydrogen, R^{3D} represents hydrogen, R^{6D} represents hydrogen or methylthio, and R^{7D} represents difluoromethanesulfonyl, or

(ii) R^{1D} represents methyl, R^{2D} represents hydrogen or fluorine, R^{3D} represents hydrogen, R^{6D} represents hydrogen, and R^{7D} represents difluoromethanesulfonyl,

and
compounds of the formula (XV)

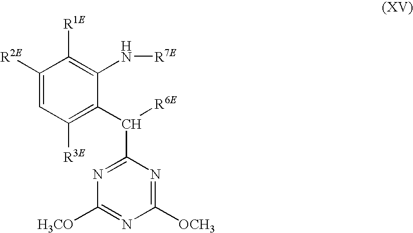

wherein

R^{1E} represents methyl, ethyl or methoxy,

R^{2E} represents hydrogen or fluorine,

R^{3E} represents hydrogen,

R^{6E} represents hydrogen or methylthio, and

R^{7E} represents hydrogen or difluoromethanesulfonyl.

The reaction of the preparation method (a) may be carried out in a suitable diluent and examples thereof include:

organic acids such as acetic acid.

The preparation method (a) can be carried out practically in a wide temperature range.

The reaction can be generally carried out at a temperature in a range of about 15° C. to about 120° C. and preferably in a range of about 15° C. to about 100° C.

Moreover, the reaction is preferably carried out under normal pressure, although it may be carried out under a high or reduced pressure.

In carrying out the preparation method (a), the aimed compounds can be obtained, for example, by reacting 1 mole of the compounds of the formula (II) with about 1 mole to about 5 mole of aqueous hydrogen peroxide in a diluent, for example, acetic acid.

The reaction of the above-mentioned preparation process (b) may be carried out in an suitable diluent. As examples of the diluent used in that case there can be mentioned water; aliphatic, alicyclic and aromatic hydrocarbons (may be optionally chlorinated), for example, hexane, cyclohexane, ligroine, toluene, xylene, dichloromethane, chloroform, carbon tetrachloride, 1,2-dichloroethane, chlorobenzene, etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM), etc.; ketones, for example, acetone, methyl ethyl ketone (MEK), methyl-isopropyl ketone, methyl isobutyl ketone (MIBK), etc.; nitriles, for example, acetonitrile, propionitrile, acrylonitrile, etc.; esters, for example, ethyl acetate, amyl acetate, etc.; acid amides, for example, dimethylformamide (DMF), dimethylacetamide (DMA), N-methylpyrrolidone, 1,3-dimethyl-2-imidazolidinone, hexamethylphosphoric triamide (HMPA), etc.; sulfones, sulfoxides, for example, dimethyl sulfoxide (DMSO), sulfolane, etc.; organic acids, for example, formic acid, acetic acid, trifluoroacetic acid, propionic acid, etc.; bases, for example, pyridine etc.

The preparation process (b) can be conducted in the presence of an acid catalyst and as examples of said acid catalyst there can be mentioned mineral acids, for example, hydrochloric acid, sulfuric acid, nitric acid, hydrobromic acid, sodium hydrogen sulfite, etc.; organic acids, for example, formic acid, acetic acid, trifluoroacetic acid, propionic acid, methanesulfonic acid, benzenesulfonic acid, p-toluenesulfonic acid, etc.

The preparation process (b) can be conducted in a substantially wide range of temperature. It is, however, preferable to conduct it at temperatures in the range of generally about −100° C. to about 150° C., particularly about 20° C. to about 120° C. Although said reaction is conducted desirably under normal pressure, it can be conducted optionally under elevated pressure or under reduced pressure.

In conducting the preparation process (b), the aimed compounds can be obtained, for example, by reacting 1 to 10 moles of chromium (VI) oxide to 1 mole of the compounds of the formula (III) in a diluent, for example, acetic acid.

The reaction of the preparation method (c) can be carried out in a suitable diluent and examples thereof include:

water;

aliphatic, alicyclic, and aromatic hydrocarbons which may be optionally chlorinated, such as pentane, hexane, cyclohexane, petroleum, ether, ligroin, benzene, toluene, xylene, dichloromethane, chloroform, carbon tetrachloride, 1,2-dichloroethane, and chlorobenzene, and dichlorobenzene;

ethers such as ethyl ether, methyl ethyl ether, isopropyl ether, butyl ether, dioxane, dimethoxyethane (DME), tetrahydrofuran (THF), and diethylene glycol dimethyl ether (DGM);

nitriles such as acetonitrile and propionitrile;

alcohols such as methanol, ethanol, isopropanol, butanol, and ethylene glycol;

esters such as ethyl acetate and amyl acetate;

acid amides such as dimethylformamide (DMF), dimethylacetamide (DMA), N-methylpyrrolidone, 1,3-dimethyl-2-imidazolidinone, hexamethylphosphoric triamide (HMPA);

sulfones and sulfoxides such as dimethyl sulfoxide (DMSO) and sulfolane; and

bases such as pyridine.

The preparation method (c) can be carried out practically in a wide temperature range.

The reaction can be generally carried out at a temperature in a range of about −100° C. to about 60° C. and preferably in a range of about −80° C. to about 40° C. The reaction is preferably carried out under normal pressure, but it may also be carried out under enhanced or reduced pressure.

In carrying out the preparation method (c), the aimed compounds can be obtained by reacting 1 mol of the compounds of the formula (IAc) with 0.25 mole to 2 mole of sodium borohydride in a diluent, for example, methanol.

Also in carrying out the preparation method (c), the reaction may start from compounds of the formula (III) to obtain compounds of the formula (IAc) and then the reaction may be continued without isolation and purification thereof, thereby to obtain compounds of the formula (IA).

The reaction of the above-mentioned preparation process (d) can be conducted in an appropriate diluent. As examples of the diluent used in that case there can be mentioned aliphatic, alicyclic and aromatic hydrocarbons (may be optionally chlorinated), for example, hexane, cyclohexane, ligroine, toluene, xylene, dichloromethane, chloroform, carbon tetrachloride, 1,2-dichloroethane, chlorobenzene, etc.; ethers, for example, ethyl ether, methyl ethyl ether, isopropyl ether, butyl ether, dioxane, dimethoxyethane (DME), tetrahydrofuran (THF), diethylene glycol dimethyl ether (DGM), etc.; ketones, for example, acetone, methyl ethyl ketone (MEK), methyl-isopropyl ketone, methyl isobutyl ketone (MIBK), etc.; nitriles, for example, acetonitrile, propionitrile, acrylonitrile, etc.; esters, for example, ethyl acetate, amyl acetate, etc.; acid amides, for example, dimethylformamide (DMF), dimethylacetamide (DMA), N-methylpyrrolidone, 1,3-dimethyl-2-imidazolidinone, hexamethylphosphoric triamide (HMPA), etc.; sulfones, sulfoxides, for example, dimethyl sulfoxide (DMSO), sulfolane, etc.; bases, for example, pyridine etc.

The preparation process (d) can be conducted in the presence of an acid binder, and as said acid binder there can be mentioned as inorganic bases, hydrides, hydroxides, carbonates, bicarbonates, etc. of alkali metals or alkaline earth metals, for example, sodium hydride, lithium hydride, sodium hydrogen carbonate, potassium hydrogen carbonate, sodium carbonate, potassium carbonate, lithium hydroxide, sodium hydroxide, potassium hydroxide, calcium hydroxide, etc.; inorganic alkali metal amides, for example, lithium amide, sodium amide, potassium amide, etc.; as organic bases, alcoholates, tertiary amines, dialkylaminoanilines and pyridines, for example, triethylamine, 1,1,4,4-tetramethylethylenediamine (TMEDA), N,N-dimethylaniline, N,N-diethylaniline, pyridine, 4-dimethylaminopyridine (DMAP), 1,4-diazabicyclo[2,2,2]octane (DABCO) and 1,8-diazabicyclo[5,4,0]undec-7-ene (DBU), etc.; organic lithium compounds, for example, methyl lithium, n-butyl lithium, sec-butyl lithium, tert-butyl lithium, phenyl lithium, dimethyl copper lithium, lithium diisopropyl amide, lithium cyclohexyl isopropyl amide, lithium dicyclohexyl amide, n-butyl lithium-DABCO, n-butyl lithium-DBU, n-butyl lithium-TMEDA, etc.

The preparation process (d) can be conducted in a substantially wide range of temperature. It is, however, preferable to conduct it at temperatures in the range of generally about −100° C. to about 130° C., particularly about −80° C. to about 130° C. Although said reaction is conducted desirably under normal pressure, it can be conducted optionally under elevated pressure or under reduced pressure.

In conducting the preparation process (d), the aimed compounds can be obtained, for example, by reacting 1 to 5 moles of the compounds of the formula (IV) to 1 mole of the compound of the formula (IAd) in a diluent, for example, acetonitrile, in the presence of 2 to 5 moles of potassium carbonate.

The reaction of the preparation method (e) can be carried out under the same conditions as the preparation method of (d).

The reaction of the preparation method (f) can be carried out under the same conditions as the preparation method of (c).

The reaction of the preparation method (g) can be carried out in a suitable diluent and examples thereof include:

in the case of using, as the halogenating agent, a fluorinating agent such as diethylaminesulfur trifluoride,

aliphatic, alicyclic, and aromatic hydrocarbons which may be optionally chlorinated, such as hexane, cyclohexane, ligroin, toluene, xylene, dichloromethane, chloroform, carbon tetrachloride, 1,2-dichloroethane, and chlorobenzene; and

ethers such as ethyl ether, methyl ethyl ether, isopropyl ether, butyl ether, dioxane, dimethoxyethane (DME), tetrahydrofuran (THF), diethylene glycol dimethyl ether (DGM); and

in the case of using, as the halogenating agent, a chlorinating agent such as phosphorus oxychloride and thionyl chloride, aliphatic, alicyclic, and aromatic hydrocarbons which may be optionally chlorinated such as hexane, cyclohexane, ligroin, benzene, toluene, xylene, dichloromethane, chloroform, carbon tetrachloride, 1,2-dichloroethane, and chlorobenzene;

ethers such as ethyl ether, methyl ethyl ether, isopropyl ether, butyl ether, dioxane, dimethoxyethane (DME), tetrahydrofuran (THF), diethylene glycol dimethyl ether (DGM); and

in the case of using, as the halogenating agent, a chlorinating agent such as phosphorus oxychloride and thionyl chloride, aliphatic, alicyclic, and aromatic hydrocarbons which may be optionally chlorinated such as hexane, cyclohexane, ligroin, benzene, toluene, xylene, dichloromethane, chloroform, carbon tetrachloride, 1,2-dichloroethane, and chlorobenzene;

ethers such as ethyl ether, methyl ethyl ether, isopropyl ether, butyl ether, dioxane, dimethoxyethane (DME), tetrahydrofuran (THF), diethylene glycol dimethyl ether (DGM); and

acid amides such as dimethylformamide (DMF), dimethylacetamide (DMA), N-methylpyrrolidone, 1,3-dimethyl-2-imidazolidinone, and hexamethylphosphoric triamide (HMPA).

The preparation method (g) can be carried out practically in a wide temperature range.

In the case of using the fluorinating agent as the halogenating agent, generally, the reaction can be carried out at a temperature in a range of about −100° C. to about 30° C. and preferably in a range of about −80° C. to about 30° C. The reaction is preferably carried out under normal pressure, but it may also be carried out under enhanced or reduced pressure.

In the case of using the chlorinating agent as the halogenating agent, generally, it can be carried out at about −100° C. to about 130° C. and preferably at about −80° C. to about 130° C. The reaction is preferably carried out under normal pressure, but it may also be carried out under enhanced or reduced pressure.

In carrying out the preparation method (g), the aimed compounds can be obtained by reacting 1 mol of the compounds of the formula (ICg) with 1 mole to 5 mole of diethylaminesulfur trifluoride in a diluent, for example, dichloromethane.

In carrying out the preparation method (g), an objective compound can be obtained by reacting 1 mole of the compounds of the formula (ICg) with 1 mole or more of thionyl chloride, which can also be used as a solvent, in a diluent, for example, dichloromethane.

The active compounds of the formula (I), according to the present invention, show excellent herbicidal activity to various kinds of weeds and can be used as herbicides, as will be set forth in Biological Test Examples below. In this specification, weeds are intended to broadly include all kinds of plant species grown in undesired places. The compounds of the formula (I), according to the present invention, works as a selective herbicide depending on the concentration thereof at the time of use. The active compounds, according to the present invention, can be used against the following weeds grown among the following cultivated plants.

Genera of weeds in Dicotyledoneae: Sinapis, Capsella, Leipidium, Galium, Stellaria, Chenopodium, Kochia, Urtica, Senecio, Amaranthus, Portulaca, Xanthium, Ipomoea, Polygonum, Ambrosia, Cirsium, Sonchus, Solanum, Rorippa, Lamium, Veronica, Datura, Viola, Galeopsis, Papaver, Centaurea, Galinsoga, Rotala, Lindernia, Sesbania, Trifolium, Abutilon, Lamium, Matricaria, Artemisia, Sesbania, Pharbitis and the like.

Genera of cultivar plants in Dicotyledoneae: Gossypium, Glycine, Beta, Daucus, Phaseolus, Pisum, Solanum, Linum, Ipomoea, Vicia, Nicotiana, Lycopersicon, Arachis, Brassica, Lactuca, Cucumis, Cucurbita, and the like.

Genera of weeds in Monocotyledoneae: Echinochlona, Setaria, Panicum, Digitaria, Phleum, Poa, Festuca, Eleusine, Lolium, Bromus, Avena, Cyperus, Sorghum, Agropyron, Monochoria, Fimbristylis, Sagittaria, Eleocharis, Scirpus, Paspalum, Ischaemum, Agrostis, Alopecurus, Cynodon, Commelina, Brachiaria, Leptochloa, and the like.

Genera of cultivar plants of Monocotyledoneae: Oryza, Zea, Triticum, Hordeum, Avena, Secale, Sorghum, Panicum, Saccharum, Ananas, Asparagus, and Allium, and the like.

The active compounds of the formula (I), according to the present invention, can be used for weeds in paddy fields. Examples of the weeds in paddy fields to be prevented and eliminated by the active compounds, according to the present invention, are as follows.

Dicotyledoneae of the following genera: Polygonum, Rorippa, Rotala, Lindemia, Bidens, Dopatrium, Eclipta, Elatine, Gratiola, Lindemia, Ludwigia, Oenanthe, Ranunculus, Deinostema, and the like.

Monocotyledoneae of the following genera: Echinochloa, Panicum, Poa, Cyperus, Monochoria, Fimbristylis, Sagittaria, Eleocharis, Scirpus, Alisma, Aneilema, Blyxa, Eriocaulon, Potamogeton, Brachiaria, Leptochloa, Sphenoclea, and the like.

The active compounds of the formula (I), according to the present invention, can be used for the following representative weeds in paddy fields.

| Plant name | |
|---|---|
| (Japanese name) | Botanical name |
| Dicotyledoneae | |
| Kikashigusa | Rotala indica Koehne |
| Azena | Lindernia procumbens Philcox |
| America azena | Lindernia dubia L. Penn. |
| Azetogarashi | Lindernia angustifolia |
| Chojitade | Ludwigia prostrata Roxburgh |
| Hirumushiro | Potamogeton distinctus A. Benn |
| Mizohakobe | Elatine triandra Schk |
| Seri | Oenanthe javanica |
| Monotyledoneae | |
| Tainubie | Echinochloa oryzicola Vasing |
| Matsubai | Eleocharis acicularis L. |
| Kuroguwai | Eleocharis kuroguwai Ohwi |
| Tamagayatsuri | Cyperus difformis L. |
| Mizugayatsuri | Cyperus serotinus Rottboel |
| Hotarui | Scirpus juncoides Roxburgh |
| Konagi | Monochoria vaginalis Presl |
| Urigawa | Sagittaria pygmaea Miq |
| Heraomodaka | Alisma canaliculatum A. Br. et Bouche |
| Omodaka | Sagittaria trifolia |
| Mizuaoi | Monochoria korsakowii |
| Nikukibi | Brachiaria plantaginea |
| Azegaya | Leptochloa chinensis |

The active compounds of the formula, according to the present invention, can be used for the weeds resistant against the sulfonylurea type herbicides. For example, the active compound may be used to the weeds exemplified above.

The active compounds of the formula (I), according to the present invention, are not particularly limited for use to these grass weeds but is similarly applicable to other grass weeds.

The active compounds, according to the present invention, can be used for preventing and eliminating weeds in cultivation of perennial plants and can be used for forestation, forestation for decorative plants, orchards, grape farms, citrus orchards, nuts orchards, banana cultivar farms, coffee plantations, tea plantations, rubber plantations, oil palm plantations, cocoa plantations, small orchards, hop cultivar farms, and the like and also used for selectively preventing and eliminating weeds in plant cultivar of annual cultivas.

The active compounds, according to the present invention, can be formulated in a conventional formulation for use. The formulation forms include solutions, wettable powders, emulsions, suspensions, dusts, water-dispersible granules, tablets, granules, suspended emulsion concentrates, microcapsules in a polymer substance, and jumbo formulation-package.

These formulations may be prepared by conventionally known methods per se, for example, by mixing an active compound with a developer, i.e., a liquid or solid diluent or carrier, and if necessary, together with a surfactant, i.e., an emulsifier and/or a dispersant and/or a foaming agent.

Examples of the liquid diluent or carrier include aromatic hydrocarbons (e.g., xylene, toluene, and alkylnaphthalene), chlorinated aromatic or chlorinated aliphatic hydrocarbons (e.g., chlorobenzenes, ethylene chlorides, and methylene chloride), aliphatic hydrocarbons [e.g., paraffins (e.g., mineral oil fractions) such as cyclohexane], alcohols (e.g., butanol and glycol), ethers, esters, and ketones thereof (e.g., acetone, methyl ethyl ketone, methyl isobutyl ketone, and cyclohexanone), strongly polar solvents (e.g., dimethylformamide and dimethyl sulfoxide) and water. In the case where water is used as a developer, an organic solvent may be used as an auxiliary solvent.

Examples of the solid diluent or carrier include pulverized natural minerals (e.g., kaolin, clay, talc, chalk, quartz, attapulgite, montmorillonite, and kieselguhr), pulverized synthetic minerals (e.g., highly dispersed silicic acid, alumina, and silicates). Examples of the solid carrier for granules include pulverized and classified rocks (e.g., calcite, marble, pumice, meerschaum, and muscovite), synthesized inorganic and organic particles, fine particles of organic substances (e.g., sawdust, husks of coconuts, stems of Sorghum, and stalks of tobacco).

Examples of the emulsifying agent and/or foaming agent include nonionic and cationic emulsifying agents [e.g., polyoxyethylene fatty acid ester, polyoxyethylene fatty acid alcohol ether (e.g., alkyl aryl polyglycol ethers, alkylsulfonates, alkylsulfate, and arylsulfonates)], and hydrolysis products of albumin.

Examples of the disintegrant include lignin sulfite waste solution and methyl cellulose.

A fixing agent may be used for the formulation (dusts, granules, and emulsions) and examples thereof include carboxymethyl cellulose, natural and synthetic polymers (e.g., gum arabi, polyvinyl alcohol, and polyvinyl acetate).

A coloring agent may also be used and examples thereof include inorganic pigments (e.g., iron oxide, titanium oxide, and Prussian blue); organic dyes such as alizarine dyes, azo dyes, and metal phthalocyanine dyes; and a trace element such as metal salts of iron, manganese, boron, copper, cobalt, molybdenum, and zinc.

The formulation may contain the active compounds of the formula (I) generally in a range of 0.01 to 95% by weight and preferably in a range of 0.1 to 90% by weight.

The active compounds of the formula (I) can be used for preventing and eliminating weeds as it is or in a formulation form. The active compounds of the formula (I) may also be used in combination with a known herbicide. A mixed herbicide composition with a known herbicide may be formulated previously in a final formulation or may be formulated by tank-mixing at the time of use. Practical examples of the herbicides usable in combination with the compounds of the formula (I) in the mixed herbicide composition are as follows, which are described as common names.

Acetamide type herbicides: for example, pretilachlor, butachlor, and tenilchlor, and alachlor, etc.;

Amide type herbicides: for example, clomeprop and etobenzanide, etc.;

Benzofuran type herbicides: for example, benfuresate, etc.;

Indandione type herbicides: for example, indanofan, etc.;

Pyrazole type herbicides: for example, pyrazolate, benzofenap, and pyrazoxyfen, etc.;

Oxazinone type herbicides: for example, oxaziclomefone, etc.;

Sulfonyl urea type herbicides: for example, bensulfuron-methyl, azimsulfron, imazosulfuron, pyrazosulfuron-methyl, cyclosulfamuron, ethoxysulfuron, and halosulfuron-methyl, orthosulfamuron, flucetosulfuron etc.;

Thiocarbamate type herbicides: for example, thiobencarb, molinate, and pyributicarb, etc.;

Triazolopyrimidine type herbicides: for example, penoxsulam, flumetsulam, florasulam, etc.;

Triazine type herbicides: for example, dimethametryn and simetryn, etc.;

Pyrazolecarbonitrile type herbicides: for example, pyraclonil, etc.;

Triazole type herbicides: for example, cafenstrole, etc.;

Quinoline type herbicides: for example, quinclorac, etc.;

Isoxazole type herbicides: for example, isoxaflutole, etc.;

Dithiophosphate type herbicides: for example, anilofos, etc.;

Oxyacetamide type herbicides: for example, mefenacet and flufenacet, etc.;

Tetrazolinone type herbicides: for example, fentrazamide, etc.;

Dicarboxylmide type herbicides: for example, pentoxazone, etc.;

Oxadiazolone type herbicides: for example, oxadiargyl and oxadiazon, etc.;

Trione type herbicides: for example, sulcotrione, benzobicyclon, mesotrione and AVH301, etc.;

Phenoxypropionate type herbicides: for example, cyhalofop-butyl, etc.;

Benzoic acid type herbicides: for example, pyriminobac-methyl, bispyribac-sodium, pyriftalid and pyrimisulfan, etc.;

Diphenyl ether type herbicides: for example, chlomethoxynil and oxyfluorfen, etc.;

Pyridine dicarbothioate type herbicides: for example, dithiopyr, etc.;

Phenoxy type herbicides: for example, MCPA and MCPB, etc.;

Urea type herbicides: for example, daimuron and cumyluron, etc.;

Naphthalenedione type herbicides: for example, quinoclamin, etc.;

Isoxazolidinone type herbicides: for example, clomazone, etc.;

Imidazolinone type herbicides: for example, imazethapyr and imazamox, etc.

The above-mentioned active compounds are known herbicides disclosed in Pesticide Manual, British Crop Protect Council (2000).

The active compounds of the formula (I) may be provided with a wider range spectrum in preventing and eliminating weeds and a wider range of applicability as a selective herbicide with lessened herbicide damage, if being mixed with a herbicide safener.

Examples of the herbicide safener include the following compounds named as the common names or development codes:

AD-67, BAS-145138, benoxacor, chloquintocet-mexyl, cyometrinil, 2,4-D, DKA-24, dichlormid, dimuron, fenchlorim, fenchlorazole-ethyl, flurazole, fluxofenim, furilazole, isoxadifen-ethyl, mefenpyr-diethyl, MG-191, naphthalic anhydride, oxabetrinil, PPG-1292, and R-29148.

The herbicide safeners are also disclosed in Pesticide Manual, British Crop Protect Council (2000).

The mixed herbicide composition containing the compounds of the formula (I) and the above known herbicides may further be mixed with the above herbicide safeners. The addition lessens the herbicide damage by the composition and provides the composition with a wider range spectrum in preventing and eliminating weeds and a wider range of applicability as a selective herbicide.

Surprisingly, some herbicide mixture compositions containing the compound of the invention in combination with a known herbicide and/or herbicide safener exhibit synergetic effects.

The active compounds of the formula (I) may be used directly as it is or in the form of a formulation such as formulated liquids for spraying, emulsions, tablets, suspensions, dusts, pastes, or a granules or in the form of a further diluted formulation thereof. The active compounds, according to the present invention, can be applied in a manner of watering, spraying, atomizing, spraying granules, or the like.

The active compounds of the formula (I), according to the invention, may be used in any stage before or after sprouting of plants and may be added in soil before seeding.

The application dose of the active compounds, according to the invention, can be varied in a practically applicable range and basically differs depending on the desired effects. In the case of using the compound as a herbicide, the application dose is, for example, about 0.0001 to about 4 kg, preferably about 0.001 to about 1 kg, per hectare.

The preparation and use of the compounds, according to the invention, will be described by way of specific examples, however the present invention is not intended to be limited only to these examples.

### SYNTHESIS EXAMPLE 1

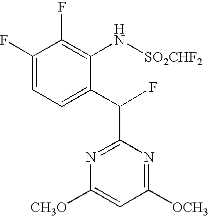

2,3-Difluoro-6-[1-(4,6-dimethoxypyrimidin-2-yl)-1-hydroxymethyl]-N-difluoromethanesulfonanilide (0.21 g, 0.51 mmol) was dissolved in dichloromethane (3 ml) and thionyl chloride (0.24 g, 2.03 mmol) was added at room temperature and the resulting solution was stirred for 4 hours. The reaction liquid was distilled in vacuo and the obtained oily product was isolated and purified with silica gel column chromatography using hexane:ethyl acetate=6:1 as elution solvent to obtain the desired 2,3-difluoro-6-[1-(4,6-dimethoxypyrimidin-2-yl)-1-chloromethyl]-N-difluoromethanesulfonanilide; (0.2 g, yield 91%).

¹H-NMR (300 MHz, CDCl₃) δ 4.01 (6H, s), 6.02 (2H, s), 6.60 (1H, t), 7.04-7.33 (2H, m), 11.31 (1H, br).

### SYNTHESIS EXAMPLE 2

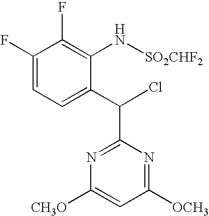

2,3-Difluoro-6-[1-(4,6-dimethoxypyrimidin-2-yl)-1-hydroxymethyl]-N-difluoromethanesulfonanilide (0.21 g, 0.51 mmol) was dissolved in dichloromethane (3 ml) and thionyl chloride (0.24 g, 2.03 mmol) was added at room temperature and the resulting solution was stirred for 4 hours. The reaction liquid was distilled in vacuo and the obtained oily product was isolated and purified with silica gel column chromatography using hexane:ethyl acetate=6:1 as elution solvent to obtain the desired 2,3-difluoro-6-[1-(4,6-dimethoxypyrimidin-2-yl)-1-chloromethyl]-N-difluoromethanesulfonanilide; (0.2 g, yield 91%).

¹H-NMR (300 MHz, CDCl₃) δ 4.01 (6H, s), 6.02 (2H, s), 6.60 (1H, t), 7.04-7.33 (2H, m), 11.31 (1H, br).

### SYNTHESIS EXAMPLE 3

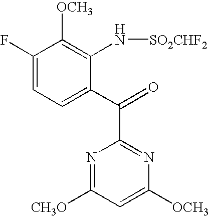

N-{6-[(4,6-dimethoxypyrimidin-2-yl)(methylthio)methyl]-3-fluoro-2-methoxyphenyl}-1,1-difluoromethanesulfonamide (705 mg, 1.56 mmol) was diluted with acetic acid (4 ml) and 31% aqueous hydrogen peroxide (205 mg) was added at room temperature. The mixture was stirred at 80° C. for 3 hours. The reaction solution was brought back to room temperature, concentrated under reduced pressure, diluted with water and then extracted three times with ethyl acetate. The organic layer was washed with water and dried. After distilling off ethyl acetate under reduced pressure, the obtained oily substance was purified by silica gel column chromatography using 1:2 mixed solvent of ethyl acetate and hexane as eluent to obtain N-{6-[(4,6-dimethoxypyrimidin-2-yl)carbonyl]-3-fluoro-2-methoxyphenyl}-1,1-difluoromethanesulfonamide (499 mg, yield 76%).

¹H NMR (CDCl₃, 300 MHz) δ 3.98 (6H, s), 4.10 (3H, s), 6.18 (1H, s), 6.70 (1H, t), 7.00 (1H, m), 7.45 (1H, m).

### SYNTHESIS EXAMPLE 4

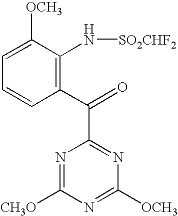

2-Methoxy-6-[(4,6-dimethoxypyrimidin-2-yl)methyl]-N-difluoromethanesulfonanilide (0.72 g, 1.84 mmol) was dissolved in acetic acid (10 ml) and chromium (VI) oxide (0.31 g, 3.05 mmol) was added thereto. The solution was heated to 80° C. and stirred for 6 hours. After stirring further 12 hours at room temperature, the reaction solution was diluted with water and extracted three times with ethyl acetate. The organic layer was washed with water. After drying, ethyl acetate was distilled off under reduced pressure, the obtained oily substance was purified by silica gel column chromatography using 2:1 mixed solvent of ethyl acetate and hexane as eluent to obtain 2-Methoxy-6-[(4,6-dimethoxypyrimidin-2-yl)carbonyl]-N-difluoromethanesulfonanilide (0.10 g, yield 13%).

H¹NMR (300 MHz, CDCl₃) δ 3.95 (3H, s), 4.10 (6H, s), 6.52 (1H, t), 7.22-7.37 (3H, m), 8.62 (1H, br).

### SYNTHESIS EXAMPLE 5

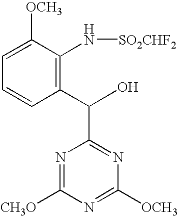

2-Methoxy-6-[(4,6-dimethoxypyrimidin-2-yl)carbonyl]-N-difluoromethane sulfonanilide (0.05 g, 0.12 mmol) was dissolved in methanol (10 ml) and, after cooling it to 5° C., sodium borohydride (0.1 g, 0.25 mmol) was added thereto while stirring. Then the solution was stirred at room temperature for 2 hours. The reaction solution was diluted with water and neutralized with citric acid. The water solution was extracted three times with ethyl acetate. After the organic layer had been washed with water and dried, ethyl acetate was distilled off under reduced pressure to obtain the objective 2-methoxy-6-[(4,6-dimethoxypyrimidin-2-yl)hydroxymethyl]-N-difluoromethanesulfonanilide (0.04 g, yield 80%).

H¹NMR (300 MHz, CDCl₃) δ 3.90 (3H, s), 4.07 (6H, s), 4.61 (1H, d), 6.11 (1H, d), 6.68 (1H, t), 6.92-6.95 (1H, m), 7.24-7.29 (2H, m), 8.62 (1H, br).

### SYNTHESIS EXAMPLE 6

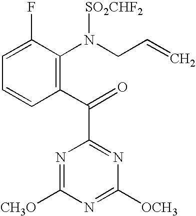

Allyl bromide (0.095 ml, 1.09 mmol) was added to a solution of 2-fluoro-6-[(4,6-dimethoxytriadin-2-yl)carbonyl-N-difluoromethanesulfonanilide (0.33 g, 0.84 mmol) and potassium carbonate (0.16 g, 1.18 mmol) in N,N-dimethylformamide (4 ml) at room temperature. The reaction mixture was stirred at room temperature for 6 hours. Ethyl acetate and water were added to the reaction mixture and the organic layer was separated and the water layer was extracted with ethyl acetate. The organic layer was dried with magnesium sulfate and evaporated to give crude product. The crude product was purified on silica gel column chromatography to give 2-fluoro-6-[(4,6-dimethoxytriadin-2-yl)carbonyl-N-(2-propenyl)-N-difluoromethanesulfonanilide (0.27 g, yield 74%).

¹H-NMR (300 MHz, CDCl₃) 4.08 (6H, s), 4.22 (1H), 4.40 (1H), 5.09-5.12 (2H), 5.80-6.00 (1H, m), 6.35 (1H, t), 7.34-7.51 (3H).

### REFERENCE EXAMPLE 1

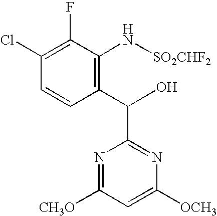

2-Fluoro-3-chloro-6-[(4,6-dimethoxypyrimidin-2-yl)carbonyl]-N-difluoromethanesulfonanilide (0.2 g, 0.47 mmol) was dissolved in methanol (5 ml) and cooled to 0° C. While stirring the solution sodium borohydride (0.04 g, 0.94 mmol) was added and then the resulting mixture was stirred at room temperature for 2 hours. The reaction liquid was distilled in vacuo, water and ether were added to the residue and the water was separated.

The obtained water layer was acidified with diluted hydrochloric acid and extracted with ethyl acetate. The obtained organic layer was washed with water, dried, and ethyl acetate was distilled in vacuo to obtain the desired 2-fluoro-3-chloro-6-[1-(4,6-dimethoxypyrimidin-2-yl)-1-hydroxymethyl]-N-difluoromethanesulfonanilide; (0.2 g, yield 99%).

¹H-NMR (300 MHz, CDCl₃) δ 4.00 (6H, s), 4.96 (1H, d), 6.02 (1H, s), 6.09 (1H, d), 6.59 (1H, t), 7.32-7.50 (2H, m), 10.73 (1H, br).

### REFERENCE EXAMPLE 2

### Alternative Method

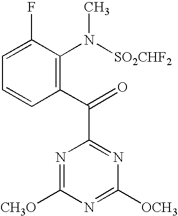

To a solution of 2-fluoro-6-[1-(4,6-dimethoxypyrimidin-2-yl)-1-methylthiomethyl]-N-difluoromethanesulfonanilide (1.96 g, 4.62 mmol) in N,N-dimethylformamide (20 ml) was added potassium carbonate (1.95 g, 13.85 mmol) and while stirring the solution at room temperature methyl iodide (0.86 ml, 13.85 mmol) was added. Then, the resulting mixture was stirred at room temperature for 48 hours. Ethyl acetate and water were added to the reaction liquid and the organic layer was separated and the water layer was further extracted with ethyl acetate. The obtained organic layer was washed with water, dried, and ethyl acetate was distilled in vacuo. The obtained oily product was isolated and purified with silica gel column chromatography using a mixed solvent of hexane:ethyl acetate=3:2 as elution solvent to obtain the desired 2-fluoro-6-[4,6-dimethoxytriazin-2-yl]carbonyl]-N-methyl-N-difluoromethanesulfonanilide; (1.08 g, yield 57.5%) as white crystal (m.p. 104-107° C.).

¹H-NMR (300 MHz, CDCl₃) δ 3.26 (3H, s), 4.12 (6H, s), 6.21 (1H, t), 7.39-7.45 (1H, m), 7.50-7.57 (1H, m), 7.64-7.67 (1H, m).

The compounds of the formula (I) obtained in the same manner as in the above Synthesis Examples 1 to 6 and Reference Examples 1 and 2 are shown in Table 1 together with the compounds synthesized in Synthesis Examples 1 to 6 and the compounds synthesized in Reference Examples 1 and 2, and the physiochemical properties thereof are shown in Table 2.

In the Table 1, OCH₂cPr represents cyclopropylmethyloxy, and (E) and (Z) represent geometrical isomerism by E,Z-nomenclature.

TABLE 1

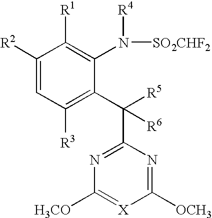

| Compound | | | | | | | |
|---|---|---|---|---|---|---|---|
| No. | R¹ | R² | R³ | R⁴ | R⁵ | R⁶ | X |
| 1 | F | H | H | CH₃ | C═O | | N |
| 2 | Cl | H | H | CH₃ | C═O | | N |
| 3 | F | F | H | H | H | OH | CH |
| 4 | F | Cl | H | H | H | OH | CH |
| 5 | H | F | H | H | H | OH | CH |
| 6 | H | H | F | H | H | OH | CH |
| 7 | F | H | F | H | H | OH | CH |
| 8 | F | F | H | H | H | OH | N |
| 9 | F | H | H | CH₃ | H | OH | N |
| 10 | F | H | H | H | H | F | CH |
| 11 | Cl | H | H | H | H | F | CH |
| 12 | F | F | H | H | H | F | CH |
| 13 | F | H | H | H | H | Cl | CH |
| 14 | Cl | H | H | H | H | Cl | CH |
| 15 | F | F | H | H | H | Cl | CH |
| 16 | F | H | H | C₂H₅ | C═O | | N |
| 17 | F | H | H | C₃H₇-n | C═O | | N |
| 18 | F | H | H | C₄H₉-n | C═O | | N |
| 19 | F | H | H | CH₂CH═CH₂ | C═O | | N |
| 20 | F | H | H | 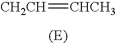 | C═O | | N |
| 21 | F | H | H | 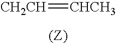 | C═O | | N |
| 22 | F | H | H | propargyl | C═O | | N |
| 23 | F | H | H | 2-butynyl | C═O | | N |
| 24 | F | H | H | CH₂OCH₃ | C═O | | N |
| 25 | F | H | H | CH₂OCH₂CH₃ | C═O | | N |
| 26 | Cl | H | H | C₂H₅ | C═O | | N |
| 27 | Cl | H | H | C₃H₇-n | C═O | | N |
| 28 | Cl | H | H | C₄H₉-n | C═O | | N |
| 29 | Cl | H | H | CH₂CH═CH₂ | C═O | | N |
| 30 | Cl | H | H | 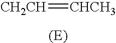 | C═O | | N |
| 31 | Cl | H | H | 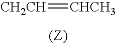 | C═O | | N |
| 32 | Cl | H | H | propargyl | C═O | | N |
| 33 | Cl | H | H | 2-butynyl | C═O | | N |
| 34 | Cl | H | H | CH₂OCH₃ | C═O | | N |
| 35 | Cl | H | H | CH₂OCH₂CH₃ | C═O | | N |
| 36 | F | H | H | C₂H₅ | H | OH | N |
| 37 | F | H | H | C₃H₇-n | H | OH | N |
| 38 | F | H | H | C₄H₉-n | H | OH | N |
| 39 | F | H | H | CH₂CH═CH₂ | H | OH | N |
| 40 | F | H | H | 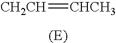 | H | OH | N |
| 41 | F | H | H | 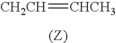 | H | OH | N |
| 42 | F | H | H | propargyl | H | OH | N |
| 43 | F | H | H | 2-butynyl | H | OH | N |
| 44 | F | H | H | CH₂OCH₃ | H | OH | N |
| 45 | F | H | H | CH₂OCH₂CH₃ | H | OH | N |
| 46 | Cl | H | H | C₂H₅ | H | OH | N |
| 47 | Cl | H | H | C₃H₇-n | H | OH | N |
| 48 | Cl | H | H | C₄H₉-n | H | OH | N |
| 49 | Cl | H | H | CH₂CH═CH₂ | H | OH | N |
| 50 | Cl | H | H | 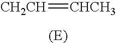 | H | OH | N |
| 51 | Cl | H | H | 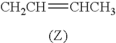 | H | OH | N |
| 52 | Cl | H | H | propargyl | H | OH | N |
| 53 | Cl | H | H | 2-butynyl | H | OH | N |
| 54 | Cl | H | H | CH₂OCH₃ | H | OH | N |
| 55 | Cl | H | H | CH₂OCH₂CH₃ | H | OH | N |
| 56 | CH₃ | H | H | H | C═O | | N |
| 57 | C₂H₅ | H | H | H | C═O | | N |
| 58 | CH₃ | F | H | H | C═O | | N |
| 59 | CH₃ | H | H | CH₃ | C═O | | N |
| 60 | CH₃ | H | H | C₂H₅ | C═O | | N |
| 61 | CH₃ | H | H | C₃H₇-n | C═O | | N |
| 62 | CH₃ | H | H | C₄H₉-n | C═O | | N |
| 63 | CH₃ | H | H | CH₂CH═CH₂ | C═O | | N |
| 64 | CH₃ | H | H | 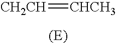 | C═O | | N |
| 65 | CH₃ | H | H | 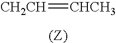 | C═O | | N |
| 66 | CH₃ | H | H | propargyl | C═O | | N |
| 67 | CH₃ | H | H | 2-butynyl | C═O | | N |
| 68 | CH₃ | H | H | CH₂OCH₃ | C═O | | N |
| 69 | CH₃ | H | H | CH₂OCH₂CH₃ | C═O | | N |
| 70 | CH₃ | F | H | H | H | OH | CH |
| 71 | CH₃ | Cl | H | H | H | OH | CH |
| 72 | CH₃ | H | F | H | H | OH | CH |
| 73 | CH₃ | H | H | H | H | OH | N |
| 74 | C₂H₅ | H | H | H | H | OH | N |
| 75 | CH₃ | F | H | H | H | OH | N |
| 76 | CH₃ | H | H | CH₃ | H | OH | N |
| 77 | CH₃ | H | H | C₂H₅ | H | OH | N |
| 78 | CH₃ | H | H | C₃H₇-n | H | OH | N |
| 79 | CH₃ | H | H | C₄H₉-n | H | OH | N |
| 80 | CH₃ | H | H | CH₂CH═CH₂ | H | OH | N |
| 81 | CH₃ | H | H | 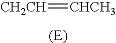 | H | OH | N |
| 82 | CH₃ | H | H | 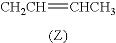 | H | OH | N |
| 83 | CH₃ | H | H | propargyl | H | OH | N |
| 84 | CH₃ | H | H | 2-butynyl | H | OH | N |
| 85 | CH₃ | H | H | CH₂OCH₃ | H | OH | N |
| 86 | CH₃ | H | H | CH₂OCH₂CH₃ | H | OH | N |
| 87 | OCH₃ | H | H | H | C═O | | CH |
| 88 | OCH₃ | F | H | H | C═O | | CH |
| 89 | OCH₃ | Cl | H | H | C═O | | CH |
| 90 | OCH₃ | H | F | H | C═O | | CH |
| 91 | OC₂H₅ | H | H | H | C═O | | CH |
| 92 | OC₃H₇-n | H | H | H | C═O | | CH |
| 93 | OC₄H₉-n | H | H | H | C═O | | CH |
| 94 | OC₃H₇-iso | H | H | H | C═O | | CH |
| 95 | OCH₂cPr | H | H | H | C═O | | CH |
| 96 | OC₄H₉-iso | H | H | H | C═O | | CH |
| 97 | OCH₃ | H | H | CH₃ | C═O | | CH |
| 98 | OCH₃ | H | H | C₂H₅ | C═O | | CH |
| 99 | OCH₃ | H | H | C₃H₇-n | C═O | | CH |
| 100 | OCH₃ | H | H | C₄H₉-n | C═O | | CH |
| 101 | OCH₃ | H | H | CH₂CH═CH₂ | C═O | | CH |
| 102 | OCH₃ | H | H | 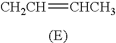 | C═O | | CH |
| 103 | OCH₃ | H | H | 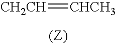 | C═O | | CH |
| 104 | OCH₃ | H | H | propargyl | C═O | | CH |
| 105 | OCH₃ | H | H | 2-butynyl | C═O | | CH |
| 106 | OCH₃ | H | H | CH₂OCH₃ | C═O | | CH |
| 107 | OCH₃ | H | H | CH₂OCH₂CH₃ | C═O | | CH |
| 108 | OC₂H₅ | H | H | CH₃ | C═O | | CH |
| 109 | OC₂H₅ | H | H | C₂H₅ | C═O | | CH |
| 110 | OC₂H₅ | H | H | C₃H₇-n | C═O | | CH |
| 111 | OC₂H₅ | H | H | C₄H₉-n | C═O | | CH |
| 112 | OC₂H₅ | H | H | CH₂OCH₃ | C═O | | CH |
| 113 | OC₂H₅ | H | H | CH₂OCH₂CH₃ | C═O | | CH |
| 114 | OCH₃ | H | H | H | C═O | | N |
| 115 | OCH₃ | H | H | CH₃ | C═O | | N |
| 116 | OCH₃ | H | H | C₂H₅ | C═O | | N |
| 117 | OCH₃ | H | H | C₃H₇-n | C═O | | N |
| 118 | OCH₃ | H | H | C₄H₉-n | C═O | | N |
| 119 | OCH₃ | H | H | CH₂CH═CH₂ | C═O | | N |
| 120 | OCH₃ | H | H | 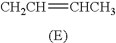 | C═O | | N |
| 121 | OCH₃ | H | H | 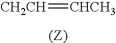 | C═O | | N |
| 122 | OCH₃ | H | H | propargyl | C═O | | N |
| 123 | OCH₃ | H | H | 2-butynyl | C═O | | N |
| 124 | OCH₃ | H | H | CH₂OCH₃ | C═O | | N |
| 125 | OCH₃ | H | H | CH₂OCH₂CH₃ | C═O | | N |
| 126 | OCH₃ | H | H | H | H | OH | CH |
| 127 | OCH₃ | F | H | H | H | OH | CH |
| 128 | OCH₃ | Cl | H | H | H | OH | CH |
| 129 | OCH₃ | H | F | H | H | OH | CH |
| 130 | OC₂H₅ | H | H | H | H | OH | CH |
| 131 | OC₃H₇-n | H | H | H | H | OH | CH |
| 132 | OC₄H₉-n | H | H | H | H | OH | CH |
| 133 | OC₃H₇-iso | H | H | H | H | OH | CH |
| 134 | OCH₂cPr | H | H | H | H | OH | CH |
| 135 | OC₄H₉-iso | H | H | H | H | OH | CH |
| 136 | OCHF₂ | H | H | H | H | OH | CH |
| 137 | OCH₃ | H | H | CH₃ | H | OH | CH |
| 138 | OCH₃ | H | H | C₂H₅ | H | OH | CH |
| 139 | OCH₃ | H | H | C₃H₇-n | H | OH | CH |
| 140 | OCH₃ | H | H | C₄H₉-n | H | OH | CH |
| 141 | OCH₃ | H | H | CH₂CH═CH₂ | H | OH | CH |
| 142 | OCH₃ | H | H | 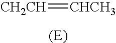 | H | OH | CH |
| 143 | OCH₃ | H | H | 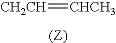 | H | OH | CH |
| 144 | OCH₃ | H | H | propargyl | H | OH | CH |
| 145 | OCH₃ | H | H | 2-butynyl | H | OH | CH |
| 146 | OCH₃ | H | H | CH₂OCH₃ | H | OH | CH |
| 147 | OCH₃ | H | H | CH₂OCH₂CH₃ | H | OH | CH |
| 148 | OC₂H₅ | H | H | CH₃ | H | OH | CH |
| 149 | OC₂H₅ | H | H | C₂H₅ | H | OH | CH |
| 150 | OC₂H₅ | H | H | C₃H₇-n | H | OH | CH |
| 151 | OC₂H₅ | H | H | C₄H₉-n | H | OH | CH |
| 152 | OC₂H₅ | H | H | CH₂OCH₃ | H | OH | CH |
| 153 | OC₂H₅ | H | H | CH₂OCH₂CH₃ | H | OH | CH |
| 154 | OCH₃ | H | H | H | H | OH | N |
| 155 | OCH₃ | H | H | CH₃ | H | OH | N |
| 156 | OCH₃ | H | H | C₂H₅ | H | OH | N |
| 157 | OCH₃ | H | H | C₃H₇-n | H | OH | N |
| 158 | OCH₃ | H | H | C₄H₉-n | H | OH | N |
| 159 | OCH₃ | H | H | CH₂CH═CH₂ | H | OH | N |
| 160 | OCH₃ | H | H | 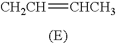 | H | OH | N |
| 161 | OCH₃ | H | H | 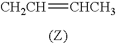 | H | OH | N |
| 162 | OCH₃ | H | H | propargyl | H | OH | N |
| 163 | OCH₃ | H | H | 2-butynyl | H | OH | N |
| 164 | OCH₃ | H | H | CH₂OCH₃ | H | OH | N |
| 165 | OCH₃ | H | H | CH₂OCH₂CH₃ | H | OH | N |
| 166 | Cl | H | H | CH₃ | H | OH | N |

TABLE 2

| Compound | Physical value (¹H-NMR (300 MHz, CDCl₃) δ) or |
|---|---|
| No. | melting point (° C.) |
| 1 | 3.26 (3H, s), 4.12 (6H, s), 6.21 (1H, t), 7.39-7.45 (1H, m), |
| | 7.50-7.57 (1H, m), 7.64-7.67 (1H, m). |
| 2 | 2.98 (3H, s), 4.12 (6H, s), 6.25 (1H, t), 7.48 (1H, t), 7.66-7.74 (2H). |
| 3 | 4.00 (6H, s), 4.96 (1H, br), 6.01 (1H, s), 6.06 (1H, s), 6.57 (1H, t), |
| | 7.13 (1H, m), 7.49 (1H, m), 10.76 (1H, br). |
| 4 | 4.00 (6H, s), 4.96 (1H, d), 6.02 (1H, s), 6.09 (1H, d), 6.59 (1H, t), |
| | 7.32-7.50 (2H, m), 10.73 (1H, br). |
| 5 | 96-99 |
| 6 | 3.92 (6H, s) 5.96 (1H, s) 6.22 (1H, s) 6.25 (1H, br t, J = 54 Hz) |
| | 6.94 (1H, br t, J = 9.0 Hz) 7.26 (1H, m) 7.37 (1H, d, J = 6.0 Hz). |
| 7 | 3.93 (6H, s), 5.99 (1H, s), 6.20 (1H, s), 6.68 (1H, t), 6.97-7.14 (2H, m). |
| 8 | 4.08 (6H, s), 6.02 (1H, s), 6.54 (1H, t), 7.13 (1H, m), 7.44 (1H, m). |
| 9*¹ | A: 3.54 (3H, s), 4.04 (6H, s), 4.47 (1H, d), 6.00 (1H, d), 6.36 (1H, t), |
| | 7.05-7.41 (3H, m). |
| | B: 3.36 (3H, s), 4.06 (6H, s), 4.46 (1H, d), 5.93 (1H, d), 6.77 (1H, t), |
| | 7.05-7.41 (3H, m). |
| 10 | 4.00 (6H, s), 6.01 (1H, s), 6.40-6.76 (2H, m), 7.15-7.41 (3H, m), |
| | 10.50 (1H, br). |
| 11 | 3.99 (6H, s), 6.00 (1H, s), 6.57-6.93 (2H, m), 7.32 (1H, t), 7.48 (1H, d), |
| | 7.57 (1H, d). |
| 12 | 4.00 (6H, s), 6.02 (1H, s), 6.50 (1H, d), 6.56 (1H, t), 7.13-7.19 (1H, m), |
| | 7.31-7.36 (1H, m), 10.71 (1H, br). |
| 13 | 4.01 (6H, s), 6.01 (1H, s), 6.04 (1H, s), 6.63 (1H, t), 7.16-7.37 (3H, m), |
| | 11.09 (1H, br). |
| 14 | 3.98 (6H, s), 5.97 (1H, s), 6.37 (1H, s), 6.78 (1H, t), 7.31 (1H, t), |
| | 7.47 (1H, dd), 7.75 (1H, dd), 9.66 (1H, br). |
| 15 | 4.01 (6H, s), 6.02 (2H, s), 6.60 (1H, t), 7.04-7.33 (2H, m), 11.31 (1H, br). |
| 16 | 1.70 (3h, t), 3.75 (1H, m), 3.86 (1H, m), 4.09 (6H, s), 6.37 (1H, t), |
| | 7.40-7.54 (3H). |
| 19 | 4.09 (6H, s), 4.26 (1H, dd), 4.36 (1H, dd), 5.08 (1H, s), 5.12 (1H), |
| | 5.90 (1H, m), 6.36 (1H, t), 7.34-7.53 (3H). |
| 22 | 2.31 (1H), 4.10 (6H, s), 4.48 (1H, dd), 4.57 (1H, dd), 6.43 (1H, t), |
| | 7.44-7.57 (3H). |
| 24 | 3.50 (3H, s), 4.11 (6H, s), 5.05 (2H), 6.40 (1H, t), 7.42-7.55 (3H). |
| 25 | 1.14 (3H, t), 3.68 (1H, m), 3.80 (1H, m), 4.08 (6H, s), 5.09 (2H, q), |
| | 6.40 (1H, t), 7.40-7.54 (3H). |
| 26 | 1.20 (3H, t), 3.85 (2H, q), 4.10 (6H, s), 6.57 (1H, t), 7.45 (1H, t), |
| | 7.55 (1H, dd), 7.74 (1H, dd). |
| 29 | 4.07 (6H, s), 4.35 (1H), 5.02 (1H, d), 5.10 (1H, d), 5.94 (1H, m), |
| | 6.52 (1H, t), 7.41 (1H, t), 7.53 (1H, dd), 7.68 (1H, dd). |
| 32 | 2.23 (1H), 4.10 (6H, s), 4.55 (1H), 6.61 (1H, t), 7.47 (1H, t), 7.60 (1H, dd), |
| | 7.74 (1H, dd). |
| 35 | 1.06 (3H, t), 3.65 (2H, m), 4.07 (6H, s), 5.00 (1H, d), 5.09 (1H, d), |
| | 6.55 (1H, t), 7.43 (1H, t), 7.54 (1H, dd), 7.71 (1H, dd). |
| 46*² | A: 1.38 (3H, t), 3.75-4.25 (2H, m), 4.04 (6H, s), 4.31 (1H, d), 5.95 (1H, d), |
| | 6.59 (1H, t), 7.16-7.35 (2H), 7.46-7.53 (1H). |
| | B: 1.28 (3H, t), 3.75-4.25 (2H, m), 4.04 (6H, s), 4.53 (1H, d), 5.90 (1H, d), |
| | 7.10 (1H, t), 7.16-7.35 (2H), 7.46-7.53 (1H). |
| 49*³ | A: 4.04 (6H, s), 4.34 (1H, d), 4.33-4.40 (2H), 5.00-5.55 (2H), |
| | 5.93 (1H, d), 6.20 (1H, m), 6.64 (1H, t), 7.2-7.6 (3H). |
| | B: 4.03 (6H, s), 4.60 (1H) 5.00-5.55 (2H), 5.90 (1H, d), 7.13 (1H, t), |
| | 7.2-7.6 (3H). |
| 56 | 2.58 (3H, s), 4.10 (6H, s), 6.23 (1H, t), 7.35 (1H, t), 7.54 (1H), 7.56 (1H). |
| 58 | 2.38 (3H, s), 4.00 (6H, s), 6.75 (1H, t), 7.35 (1H, t), 7.72-7.80 (2H), |
| | 9.40 (1H). |
| 59 | 2.50 (3H, s), 3.36 (3H, s), 4.10 (6H, s), 6.38 (1H, t) 7.38 (1H, t), 7.48 (1H), |
| | 7.53 (1H). |
| 69 | 1.14 (3H, t), 2.59 (3H, s), 3.56 (1H, m), 3.76 (1H, m), 4.10 (6H, s), |
| | 5.02 (1H, d), 5.16 (1H, d), 6.52 (1H, t), 7.38 (1H), 7.46 (1H), 7.57 (1H). |
| 70 | 108-114 |
| 71 | 128-134 |
| 72 | 2.39 (3H, s) 3.90 (6H, s) 5.95 (1H, s) 6.17 (1H, s) 6.67 (1H, br t, J = 54 Hz) |
| | 6.92 (1H, br dd, J = 9.0, 9.0 Hz) 7.16 (1H, br dd, J = 9.0, 6.0 Hz). |
| 73 | 2.50 (3H, s), 4.08 (6H, s), 4.60 (1H), 6.21 (1H), 6.41 (1H, t), |
| | 7.20-7.28 (2H), 7.48 (1H). |
| 74 | 1.24 (3H, t), 2.83 (1H, m), 2.96 (1H, m), 4.04 (6H, s), 4.68 (1H, d), |
| | 6.22 (1H, d), 6.45 (1H, t), 7.25-7.29 (2H), 7.43 (1H), 9.40 (1H, s). |
| 75 | 2.37 (3H, s), 4.09 (6H, s), 6.13 (1H, s), 6.41 (1H, t), 7.01 (1h, t), |
| | 7.20-7.50 (2H). |
| 76*⁴ | A: 2.47 (3H, s), 3.52 (3H, s), 4.03 (6H, s), 4.32 (1H, d), 5.82 (1H, d), |
| | 6.66 (1H, t), 6.86-7.30 (3H). |
| | B: 2.48 (3H, s), 3.92 (3H, s), 4.03 (6H, s), 4.43 (1H, d), 5.86 (1h, d), |
| | 7.0-7.30 (4H). |
| 77*⁵ | A: 1.33 (3H, t), 2.49 (3H, s), 4.02 (6H, s), 3.75-4.20 (2H, m), 4.32 (1H, d), |
| | 5.86 (1h, d), 6.74 (1H, t), 6.58 (!H), 7.18-7.30 (2H). |
| | B: 2.53 (3H, s), 3.75-4.20 (2H, m), 4.07 (3H, s9, 4.47 (1h, d), |
| | 5.84 (1H, d), 7.0-7.5 (4H). |
| 80*⁶ | A: 2.45 (3H, s), 4.04 (6H, s), 4.33 (1H, d), 4.50 (2H), 5.15-5.89 (2H), |
| | 6.78 (1H, d), 5.80-6.20 (1H), 6.74 (1H, t), 6.95 (1H), 7.20-7.25 (2H). |
| | B: 2.50 (3H, s), 4.03 (6H, s), 4.54 (1H, d), 5.15-5.89 (2H), 5.83 (1H, d), |
| | 5.80-6.20 (1H), 7.0-7.3 (m). |
| 86*⁷ | A: 1.18 (3H, t), 2.53 (3H, s), 4.0-4.8 (2H, m), 4.04 (6H, s), 4.38 (1H, d), |
| | 5.0-5.3 (2H), 5.46 (1H, d), 6.76 (1H, t), 7.0-7.5 (3H). |
| | B: 1.13 (3H, t), 2.51 (3H, s), 4.0-4.8 (2H), 4.00 (6H, s), 4.25 (1H, d), |
| | 6.80 (1H, d), 7.0-7.4. |
| 87 | 147-148 |
| 88 | 133-137 |
| 89 | 89-95 |
| 90 | 134-143 |
| 91 | 154-156 |
| 97 | 133-137 |
| 114 | 3.95 (3H, s), 4.10 (6H, s), 6.52 (1H, t), 7.22-7.37 (3H, m), 8.62 (1H, br). |
| 115 | 3.12 (3H, s), 3.96 (3H, s), 4.11 (6H, s), 6.13 (1H, t), 7.19-7.26 (1H, m), |
| | 7.42-7.53 (2H, m). |
| 126 | 123-137 |
| 127 | 106-112 |
| 128 | 125-130 |
| 129 | 140-145 |
| 130 | 144-145 |
| 166*⁸ | A: 3.56 (3H, s), 4.04 (6H, s), 4.40 (1H, d), 5.95 (1H, d), 6.51 (1H, t), |
| | 7.26-7.30 (2H), 7.42-7.50 (1H). |
| | B: 3.41 (3H, s), 4.05 (6H, s), 4.50 (1H, d), 5.90 (1H, d), 7.08 (1H, t), |
| | 7.25-7.50 (3H). |

*¹The compound No. 9 represents a mixture of rotational isomers A and B in a ratio of about 3.0:1.
*²The compound No. 46 represents a mixture of rotational isomers A and B in a ratio of about 1.9:1.
*³The compound No. 49 represents a mixture of rotational isomers A and B in a ratio of about 2.5:1.
*⁴The compound No. 76 represents a mixture of rotational isomers A and B in a ratio of about 3.2:1.
*⁵The compound No. 77 represents a mixture of rotational isomers A and B in a ratio of about 2.2:1.
*⁶The compound No. 80 represents a mixture of rotational isomers A and B in a ratio of about 6.8:1.
*⁷The compound No. 86 represents a mixture of rotational isomers A and B in a ratio of about 4.6:1.
*⁸The compound No. 166 represents a mixture of rotational isomers A and B in a ratio of about 3.0:1.

### SYNTHESIS EXAMPLE 7

### Intermediate

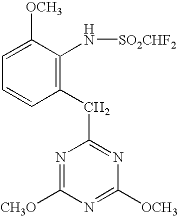

2-Methoxy-6-[(4,6-dimethoxytriazin-2-yl)methyl]aniline (0.90 g, 3.26 mmol) was dissolved in dichloromethane (3 ml) and pyridine (0.28 g, 3.58 mmol) was added thereto. The solution was cooled to −5° C. and a solution of difluoromethanesulfonyl chloride (0.54 g, 3.58 mmol) in dichloromethane (1 ml) was added thereto. The reaction solution was stirred at room temperature for two days and after addition of water it was extracted three times with dichloromethane. After the organic layer had been washed with water and dried, dichloromethane was distilled off under reduced pressure and the objective 2-Methoxy-6-[(4,6-dimethoxytriazin-2-yl)methyl]-N-difluoromethanesulfonanilide g, yield 63%) was obtained as white crystals from the obtained oily substance by silica gel column chromatography using 1:1 mixed solvent of ethyl acetate and hexane as eluent.

¹H NMR (CDCl₃, 300 MHz) δ 3.89 (3H, s), 4.04 (6H, s), 4.21 (2H, s), 6.68 (1H, t), 6.90 (1H, dd), 7.00 (1H, dd), 7.20 (1H, t), 9.86 (1H, br).

### SYNTHESIS EXAMPLE 8

### Intermediate

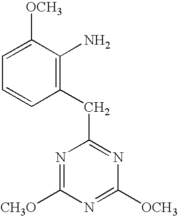

To 30 ml of a methanol solution of 2-Methoxy-6-[1-(4,6-dimethoxy -triazin-2-yl)-1-methylthiomethyl]aniline (3.20 g, 9.93 mmol) and (4.72 g, 19.85 mmol) of nickel (II) chloride hexahydrate, (1.50 g, 39.70 mmol) of sodium borohydride was added at 0° C. and the reaction solution was stirred at room temperature for 2 hours. After the reaction solution was distilled off under reduced pressure, aqueous ammonia and ethyl acetate were added and the insoluble matter was filtered off. The organic layer was separated and the water layer was further extracted three times with ethyl acetate. After the organic layer had been washed with water and drying, ethyl acetate was distilled off under reduced pressure and the objective 2-Methoxy-6-[(4,6-dimethoxypyrimidin-2-yl)methyl]aniline (1.00 g, yield 36%) was obtained from the obtained oily substance by silica gel column chromatography using 1:1 mixed solvent of ethyl acetate and hexane as eluent. ¹H NMR (CDCl₃, 300 MHz) δ 3.84 (3H, s), 3.98 (2H, s), 4.00 (6H, s), 4.71 (2H, br), 6.66-6.74 (2H, m), 6.88-6.91 (1H, m).

### SYNTHESIS EXAMPLE 9

### Intermediate

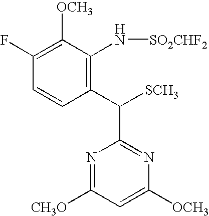

6-[(4,6-dimethoxypyrimidin-2-yl)(methylthio)methyl]-3-fluoro-2-methoxya niline (1.93 g, 5.69 mmol) was dissolved in dichloromethane (10 ml) and cooled on ice bath. Difluoromethanesulfonyl chloride (1.28 g, 8.53 mmol) and pyridine (0.90 g, 11.4 mmol) was added. The reaction solution was stirred at room temperature for 12 hours. Another difluoromethanesulfonyl chloride (0.43 g, 2.84 mmol) and pyridine (0.45 g, 5.69 mmol) was added on ice bath. The reaction solution was stirred at room temperature for 12 hours. Then saturated NH₄Cl aqueous solution was added and the mixture was extracted with dichloromethane. The organic layer was dried and distilled off under reduced pressure. The obtained oily substance was purified by silica gel column chromatography using 1:5 mixed solvent of ethyl acetate and hexane as eluent to obtain N-{6-[(4,6-dimethoxypyrimidin-2-yl)(methylthio)methyl]-3-fluoro-2-methoxyphenyl}-1,1-difluoromethanesulfonamide (0.75 g, yield 27%).

¹H NMR (CDCl₃, 300 MHz) δ 2.06 (3H, s), 3.98 (6H, s), 4.00 (3H, s), 5.25 (1H, s), 5.95 (1H, s), 6.73 (1H, t), 7.00 (1H, m), 7.35 (1H, m), 10.8 (1H, m).

### SYNTHESIS EXAMPLE 10

### Intermediate

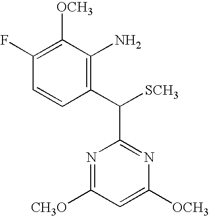

3-fluoro-2-methoxyaniline (1.28 g, 9.08 mmol) and 4,6-dimethoxy-2-[(methylthio)methyl]pyrimidine (2.00 g, 9.99 mmol) was dissolved in dichloromethane (50 ml) and the solution was cooled to −60° C. To the cooled solution tert-butyl hypochlorite (1.18 g, 10.9 mmol) was added dropwise and the solution was stirred at −60° C. for 1 hour. To the reaction solution a 28% methanol solution of sodium methoxide (3.50 g, 18.2 mmol) was added and the solution was stirred until its temperature reached room temperature in 3 hours. Saturated NH₄Cl aqueous solution was added and the mixture was extracted with dichloromethane. The organic layer was washed with water and dried. Then dichloromethane was distilled off under reduced pressure and the obtained oily substance was purified by silica gel column chromatography using 1:5 mixed solvent of ethyl acetate and hexane as eluent to obtain 6-[(4,6-dimethoxypyrimidin-2-yl)(methylthio)methyl]-3-fluoro-2-methoxyaniline (1.92 g, yield 62%).

¹H NMR (CDCl₃, 300 MHz) δ 2.04 (3H, s), 3.91 (3H, s), 3.92 (6H, s), 4.91 (2H, br s), 5.08 (1H, s), 5.90 (1H, s), 6.45 (1H, m), 7.17 (1H, m).

The compounds obtained in the same manner as in the above Synthesis Examples 7 are shown in Table 3 together with the compound synthesized in Synthesis Examples 7, and

the compounds obtained in the same manner as in the above Synthesis Examples 8 are shown in Table 4 together with the compound synthesized in Synthesis Examples 8, and
the compounds obtained in the same manner as in the above Synthesis Examples 9 are shown in Table 5 together with the compound synthesized in Synthesis Examples 9, and
the compounds obtained in the same manner as in the above Synthesis Examples 10 are shown in Table 6 together with the compound synthesized in Synthesis Examples 10, and
the physiochemical properties thereof are shown in Table 7.

In the table 3 to 6, OCH₂cPr represents cyclopropylmethyloxy.

TABLE 3

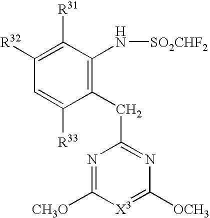

| Compound | | | | |
|---|---|---|---|---|
| No. | R³¹ | R³² | R³³ | X³ |
| 3-1 | CH₃ | Cl | H | CH |
| 3-2 | CH₃ | H | F | CH |
| 3-3 | OCH₃ | F | H | CH |
| 3-4 | OCH₃ | Cl | H | CH |
| 3-5 | OCH₃ | H | F | CH |
| 3-6 | OC₂H₅ | H | H | CH |
| 3-7 | OC₃H₇-n | H | H | CH |
| 3-8 | OC₄H₉-n | H | H | CH |
| 3-9 | OC₃H₇-iso | H | H | CH |
| 3-10 | OCH₂cPr | H | H | CH |
| 3-11 | OC₄H₉-iso | H | H | CH |
| 3-12 | CH₃ | H | H | N |
| 3-13 | C₂H₅ | H | H | N |
| 3-14 | CH₃ | F | H | N |
| 3-15 | OCH₃ | H | H | N |

TABLE 4

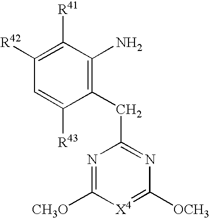

| Compound | | | | |
|---|---|---|---|---|
| No. | R⁴¹ | R⁴² | R⁴³ | X⁴ |
| 4-1 | CH₃ | F | H | CH |
| 4-2 | CH₃ | Cl | H | CH |
| 4-3 | CH₃ | H | F | CH |
| 4-4 | OCH₃ | F | H | CH |
| 4-5 | OCH₃ | Cl | H | CH |
| 4-6 | OCH₃ | H | F | CH |
| 4-7 | OC₂H₅ | H | H | CH |
| 4-8 | OC₃H₇-n | H | H | CH |
| 4-9 | OC₄H₉-n | H | H | CH |
| 4-10 | OC₃H₇-iso | H | H | CH |
| 4-11 | OCH₂cPr | H | H | CH |
| 4-12 | OC₄H₉-iso | H | H | CH |
| 4-13 | OCHF₂ | H | H | CH |
| 4-14 | CH₃ | H | H | N |
| 4-15 | C₂H₅ | H | H | N |
| 4-16 | CH₃ | F | H | N |
| 4-17 | OCH₃ | H | H | N |

TABLE 5

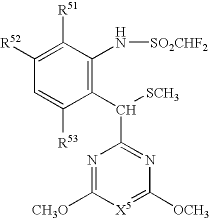

| Compound | | | | |
|---|---|---|---|---|
| No. | R⁵¹ | R⁵² | R⁵³ | X⁵ |
| 5-1 | CH₃ | F | H | CH |
| 5-2 | CH₃ | Cl | H | CH |
| 5-3 | CH₃ | H | F | CH |
| 5-4 | OCH₃ | F | H | CH |
| 5-5 | OCH₃ | Cl | H | CH |
| 5-6 | OCH₃ | H | F | CH |
| 5-7 | OC₂H₅ | H | H | CH |
| 5-8 | OC₃H₇-n | H | H | CH |
| 5-9 | OC₄H₉-n | H | H | CH |
| 5-10 | OC₃H₇-iso | H | H | CH |
| 5-11 | OCH₂cPr | H | H | CH |
| 5-12 | OC₄H₉-iso | H | H | CH |
| 5-13 | CH₃ | H | H | N |
| 5-14 | C₂H₅ | H | H | N |
| 5-15 | CH₃ | F | H | N |
| 5-16 | OCH₃ | H | H | N |

TABLE 6

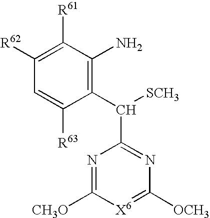

| Compound | | | | |
|---|---|---|---|---|
| No. | R⁶¹ | R⁶² | R⁶³ | X⁶ |
| 6-1 | CH₃ | F | H | CH |
| 6-2 | CH₃ | Cl | H | CH |
| 6-3 | CH₃ | H | F | CH |
| 6-4 | OCH₃ | F | H | CH |
| 6-5 | OCH₃ | Cl | H | CH |
| 6-6 | OCH₃ | H | F | CH |
| 6-7 | OC₂H₅ | H | H | CH |
| 6-8 | OC₃H₇-n | H | H | CH |
| 6-9 | OC₄H₉-n | H | H | CH |
| 6-10 | OC₃H₇-iso | H | H | CH |
| 6-11 | OCH₂cPr | H | H | CH |
| 6-12 | OC₄H₉-iso | H | H | CH |
| 6-13 | OCHF₂ | H | H | CH |
| 6-14 | CH₃ | H | H | N |
| 6-15 | C₂H₅ | H | H | N |
| 6-16 | CH₃ | F | H | N |
| 6-17 | OCH₃ | H | H | N |

TABLE 7

| Compound No. | ¹H NMR (CDCl₃, 300 MHz) δ |
|---|---|
| 3-12 | 2.48 (3H, s), 4.02 (6H, s), 4.23 (2H, s), 6.36 (1H, t), 7.12-7.25 (3H). |
| 3-13 | 1.25 (3H, t), 2.89 (2H, q), 4.02 (6H, s), 4.25 (2H, s), 6.36 (1H, t), |
| | 7.18-7.25 (3H), 9.98 (1H, s). |
| 3-14 | 2.37 (3H, s), 4.02 (6H, s), 4.18 (2H, s), 6.35 (1H, t), 6.94 (1H, t), |
| | 7.16 (1H). |
| 3-15 | 3.89 (3H, s), 4.04 (6H, s), 4.21 (2H, s), 6.68 (1H, t), 6.90 (1H, dd), |
| | 7.00 (1H, dd), 7.20 (1H, t), 9.86 (1H, br). |
| 4-14 | 2.50 (3h, s), 3.96 (2H, s), 4.01 (6H, s9, 4.51 (1H, s), 6.65 (1H, s), |
| | 6.96 (1H, d), 7.13 (1H, d). |
| 4-15 | 1.25 (3H, t), 2.51 (2h, q), 3.96 (2H, s), 4.00 (6h, s), 4.56 (2H, s), |
| | 6.70 (1H, t), 6.99 (1h, d), 7.14 (1H, d). |
| 4-16 | 2.05 (3H, s), 3.90 (3H, s), 4.01 (6H, s), 4.61 (2H, s), 6.43 (1H, t), |
| | 7.05 (1H, dd). |
| 4-17 | 3.84 (3H, s), 3.98 (2H, s), 4.00 (6H, s), 4.71 (2H, br), |
| | 6.66-6.74 (2H, m), 6.88-6.91 (1H, m). |
| 5-2 | 2.02 (3H, s), 2.50 (3H, s), 3.95 (6H, s), 5.67 (1H, s), 5.90 (1H, s), |
| | 6.46 (1H, t), 7.37 (1H, m), 7.88 (1H, m). |
| 5-3 | 2.19 (3H, s), 2.43 (3H, s), 3.95 (6H, s), 5.69 (1H, br s), 5.96 (1H, s), |
| | 6.64 (1H, t), 6.98 (1H, m), 7.19 (1H, m). |
| 5-4 | 2.06 (3H, s), 3.98 (6H, s), 4.01 (3H, s), 5.25 (1H, s), 5.95 (1H, s), |
| | 6.73 (1H, t), 7.00 (1H, m), 7.33 (1H, m), 10.8 (1H, br s). |
| 5-5 | 1.97 (3H, s), 3.86 (3H, s), 3.91 (6H, s), 5.24 (1H, s), 5.88 (1H, s), |
| | 6.70 (1H, t), 7.22 (1H, d), 7.38 (1H, d), 10.7 (1H, br s). |
| 5-6 | 2.17 (3H, s), 3.87 (3H, s), 3.99 (6H, s), 5.60 (1H, s), 5.97 (1H, s), |
| | 6.60 (1H, t), 6.93 (1H, m), 6.98 (1H, m), 11.5 (1H, m). |
| 5-7 | 1.25 (3H, t), 2.06 (3H, s), 3.97 (6H, s), 4.14 (2H, q), 5.38 (1H, s), 5.92 |
| | (1H, s), 6.87 (1H, t), 6.88 (1H, d), 7.23 (1H, m), |
| | 7.35 (1H, d), 10.3 (1H, m). |
| 6-1 | 2.02 (3H, s), 2.06 (3H, s), 3.92 (6H, s), 4.69 (2H, br s), 5.09 (1H, s), |
| | 5.89 (1H, s), 6.46 (1H, m), 7.28 (1H, m). |
| 6-2 | 2.03 (3H, s), 2.23 (3H, s), 3.92 (6H, s), 4.70 (2H, br s), 5.10 (1H, s), |
| | 5.89 (1H, s), 6.79 (1H, d), 7.30 (1H, d). |
| 6-3 | 2.05 (3H, s), 2.07 (3H, s), 3.91 (6H, s), 4.84 (2H, br s), 5.75 (1H, s), |
| | 5.90 (1H, s), 6.40 (1H, m), 6.90 (1H, m). |
| 6-4 | 2.04 (3H, s), 3.91 (3H, s), 3.92 (6H, s), 4.91 (2H, br s), 5.08 (1H, s), |
| | 5.90 (1H, s), 6.45 (1H, m), 7.17 (1H, m). |
| 6-5 | 2.04 (3H, s), 3.84 (3H, s), 3.92 (6H, s), 4.91 (2H, br s), 5.08 (1H, s), |
| | 5.90 (1H, s), 6.70 (1H, d), 7.24 (1H, d). |
| 6-6 | 2.09 (3H, s), 3.76 (3H, s), 3.91 (6H, s), 5.10 (2H, br s), 5.69 (1H, s), |
| | 5.89 (1H, s), 6.39 (1H, m), 6.61 (1H, m). |
| 6-7 | 1.42 (3H, t), 2.04 (3H, s), 3.92 (6H, s), 4.08 (2H, q), 4.77 (2H, br s), |
| | 5.19 (1H, s), 5.88 (1H, s), 6.70 (2H, m), 7.15 (1H, m). |
| 6-13 | 2.04 (3H, s), 3.93 (6H, s), 4.86 (2H, br), 5.15 (1H, s), 5.91 (1H, s), |
| | 6.45 (1H, t) 6.67-6.79 (1H, m), 6.95-6.97 (1H, m), 7.36-7.39 (1H, m) |
| 6-14 | 2.03 (3H, s), 2.14 (3H, s), 4.00 (6H, s), 5.05 (1H, s), 6.66 (1H, t), |
| | 6.98 (1H, d), 7.33 (1H, d). |
| 6-17 | 2.06 (3H, s), 3.85 (3H, s), 4.02 (6H, s), 4.62 (1H, br), 5.01 (1H, s), |
| | 6.68-6.81 (2H, m), 7.11-7.14 (1H, m). |

### REFERENCE EXAMPLE 3

### Preparation of Intermediate

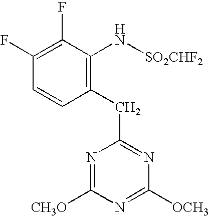

To a solution of 2,3-difluoro-6-[(4,6-dimethoxytriazin-2-yl)methyl]aniline (3.67 g, 13.0 mmol) and pyridine (1.65 g, 20.8 mmol) in methylene chloride (25 ml) was dropwise added a solution of difluoromethanesulfonyl chloride (3.13 g, 20.8 mmol) in methylene chloride (5 ml) at −30° C. or below and stirred for 1 hour. The temperature of the reaction solution was raised to room temperature and stirred for 2 days. The reaction solution was washed with water and the organic layer was dried with anhydrous magnesium sulfate and concentrated in vacuo. The obtained residue was isolated and purified with silica gel column chromatography using a solvent mixture of acetone and hexane 1:3 as elution solvent to obtain the desired 2,3-difluoro-6-[(4,6-dimethoxytriazin-2-yl)methyl]-N-difluoromethanesulfonanilide (1.54 g, yield 29.9%).

¹H-NMR (CDCl₃, 300 MHz) δ 4.05 (6H, s), 4.18 (2H, s), 6.55 (1H, t), 7.0-7.18 (2H, m).

### REFERENCE EXAMPLE 4

### Preparation of Intermediate

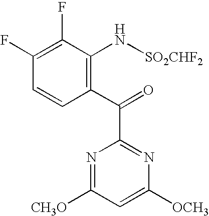

2,3-Difluoro-6-[1-(4,6-dimethoxypyrimidin-2-yl)-1-methylthiomethyl]-N-difluoromethanesulfonanilide (0.32 g, 0.73 mmol) was diluted with acetic acid (4 ml), and then 33% aqueous hydrogen peroxide (1.5 g) was added at room temperature. The mixture was stirred at room temperature for 2 hours and further stirred at 80° C. for 3 hours. The temperature of the reaction solution was reverted to room temperature and diluted with water and extracted with ethyl acetate for 3 times. The organic layer was washed with water, dried, and ethyl acetate was distilled in vacuo. The obtained oily product was isolated and purified with silica gel column chromatography using a solvent mixture of ethyl acetate and hexane 1:2 as elution solvent to obtain the desired 2,3-difluoro-6-[(4,6-dimethoxypyrimidin-2-yl)carbonyl]-N-difluoromethanesulfonanilide; (0.25 g, yield 84.3%).

¹H-NMR (CDCl₃, 300 MHz) δ 3.98 (6H, s), 4.74 (2H, s), 6.21 (1H, s), 6.51 (1H, t), 7.14 (1H, m), 7.61 (1H, m).

### Comparative Compound

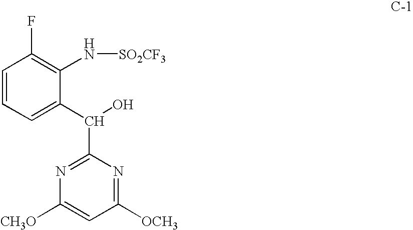

(C-1 is an analogous compound disclosed in WO96/41799)

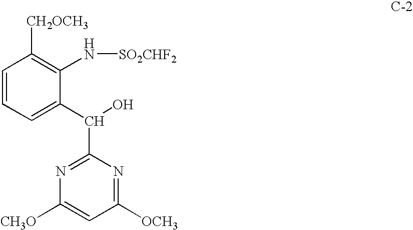

(C-2 is an analogous compound disclosed in Japanese Patent Application Laid-Open (KOKAI) No. 2000-44546.)

C-3 Ethoxysulfuron (common name)

C-4 Bensulfuron-methyl (common name)

### BIOLOGICAL TEST EXAMPLE 1

### Herbicidal Efficacy Test to Weeds Resistant to Sulfonylurea Type Herbicide

Preparation of formulation of active compound

Carrier: DMF 5 parts by weight

Emulsifier: benzyloxypolyglycol ether 1 part by weight

The formulations of active compounds were obtained as emulsions by mixing 1 part by weight of the active compound with the carrier and emulsifier in the described amounts. The formulations were diluted with water so as to adjust a prescribed dosage.

In a greenhouse respective seeds of Scirpus juncoides Roxburgh (from Hokkaido, Japan) and Lindernia procumbens Philcox (from Saitama, Japan), which are confirmed to have resistance to a sulfonylurea type herbicide, were inoculated in pots filled with 500 cm² of paddy field soil and water was poured to the pot in about 2 to 3 cm depth. At the beginning of sprouting of the weeds, prescribed diluted solutions of the formulations of the respective active compounds obtained by the above-mentioned manner were applied on the water surface. After the treatment water depth of 3 cm was kept. The herbicidal effect was investigated after 3 weeks of the treatment. The herbicidal effect was rated as 100% in the case of complete withering and 0% in the case of no herbicidal effect. When the herbicidal effect was 80% or higher, the practical effectiveness as herbicides is recognized. As the representative examples, the test results of the compound Nos. 1, 3, 4, 5, 6, 7, 13, 70, 127, 128 and 130 are shown in Table 8.

TABLE 8

| | Herbicidal effect to | |
|---|---|---|
| | sulfonylurea resistant weeds | |
| | Scirpus juncoides | Lindernia procumbens |
| Dosage | Roxburgh | Philcox |
| (g ai/ha) | (from Hokkaido) | (from Saitama pref.) |

| | | | |
|---|---|---|---|
| Compound | | | |
| No. | | | |
| 1 | 60 | 100 | — |
| 3 | 60 | 100 | 100 |
| 4 | 60 | 100 | 100 |
| 5 | 60 | 100 | 100 |
| 6 | 60 | 100 | 100 |
| 7 | 60 | 100 | 100 |
| 13 | 60 | 100 | 100 |
| 70 | 60 | 100 | 90 |
| 127 | 60 | 100 | 90 |
| 128 | 60 | 90 | 100 |
| 130 | 60 | 100 | 100 |
| Control | | | |
| C-3 | 60 | 10 | 10 |
| C-4 | 60 | 10 | 10 |

Note:
ai = active ingredient = active compound

### BIOLOGICAL TEST EXAMPLE 2

### Herbicide Damage on Transplanted Paddy Rice

In a greenhouse each three paddy rice seedlings (cultivar: Nihonbare) were transplanted (transplantation depth 2 cm) in each pot filled with 500 cm² of paddy field soil and covered with water in about 2 to 3 cm depth. After 5 days from the transplantation, prescribed diluted solutions of the formulations of the respective active compounds obtained in the same manner as in Test Example 1 were applied to the water surface of each pot. After the treatment, the water depth of 3 cm was kept. The herbicide damage was investigated after 3 weeks of the treatment. The herbicide damage was rated as 100% in the case of complete withering and 0% in the case of no herbicide damage. When the herbicide damage was 20% or lower, the safety as a paddy rice herbicide was evaluated to be excellent. As the representative examples, the test results of the compound Nos. 3, 4, 5, 6, 7, 70, 115, 127 and 128, and comparative compound C-2 are shown in Table 9.

TABLE 9

| | | Herbicide damage on |
|---|---|---|
| | Dosage (g ai/ha) | transplanted paddy rice |
| Compound No. | | |
| 3 | 60 | 10 |
| 4 | 60 | 10 |
| 5 | 60 | 0 |
| 6 | 60 | 10 |
| 7 | 60 | 10 |
| 70 | 60 | 10 |
| 115 | 60 | 0 |
| 127 | 60 | 0 |
| 128 | 60 | 0 |
| Comparison | | |
| C-2 | 60 | 30 |

### BIOLOGICAL TEST EXAMPLE 3

### Safety and Herbicidal Effect on Irrigated Directly Seeded Paddy Rice

In a greenhouse seeds of rice (cultivar: Balilla) and weeds (Brachiaria plantaginea, Cyperus esculntus, Cyperus iria L., Echinochloa colonum, Leptochloa chinensis, Ipomoea purpurea, and Sesbania exaltata) were inoculated on the surface layers of pots filled with 100 cm² of paddy field soil and covered with soil. Water was poured to produce wet state (water level 0 cm). The prescribed diluted solutions of the formulations of the respective active compounds obtained in the same manner as in Test Example 1 were sprayed on the soil of some pots on completion of the seeding and the solutions of the agents were sprayed over the plants from the above in the rest of the pots after the respective sample plants were grown in first- to third-1 eaf stage in the greenhouse. After one day from the treatment with the compound, water was poured in 3 cm depth. The herbicidal effects and herbicide damage on rice of the respective compounds were investigated after 3 weeks from the treatment. The herbicidal effect and herbicide damage on the rice were rated as 100% in the case of complete withering and 0% in the case of no herbicidal effect or no harm. When herbicidal effect was 80% or higher, it was determined to be practically applicable as herbicides. When the herbicide damage was 20% or lower, the safety of the herbicide was evaluated to be excellent. As the representative example, the test results of the compound No. 1, 2 and 9 are shown in Tables 10 and 11.

TABLE 10
Spraying to soil before sprouting

| Comp. | dosage | | Brachiaria | Cyperus | |
|---|---|---|---|---|---|
| No. | (g ai/ha) | Rice | plantaginea | esculntus | Cyperus iria L. |
| 1 | 20 | 20 | 80 | 100 | 100 |
| 2 | 20 | 30 | 80 | 90 | 100 |
| 9 | 20 | 20 | 70 | 100 | 100 |

| Comp. | Dosage | Echinochloa | Leptochloa | Ipomoea | Sesbania |
|---|---|---|---|---|---|
| No. | (g ai/ha) | colonum | chinensis | purpurea | exaltata |
| 1 | 20 | 100 | 100 | 90 | 100 |
| 2 | 20 | 80 | 90 | 80 | 80 |
| 9 | 20 | 100 | 80 | 70 | 100 |

TABLE 11
Spraying to stems and leaves after sprouting

| | dosage | | Brachiaria | Cyperus | |
|---|---|---|---|---|---|
| Comp. No. | (g ai/ha) | Rice | plantaginea | esculntus | Cyperusiria L. |
| 1 | 20 | 20 | 100 | 100 | 100 |
| 2 | 20 | 10 | 100 | 100 | 100 |
| 9 | 20 | 20 | 100 | 100 | 100 |

| Comp. | Dosage | Echinochloa | Leptochloa | Ipomoea | Sesbania |
|---|---|---|---|---|---|
| No. | (g ai/ha) | colonum | chinensis | purpurea | exaltata |
| 1 | 20 | 100 | 80 | 90 | 100 |
| 2 | 20 | 100 | — | 100 | 100 |
| 9 | 20 | 100 | 60 | 100 | 100 |

### BIOLOGICAL TEST EXAMPLE 4

### Herbicidal Effect on Weeds in Dry Field and Herbicide Damage to Crops in Dry Field (Spraying Treatment on Soil Before Sprouting)

In a greenhouse one seed each of crops in dry field (Triticum aestivum and Glycine max) and weeds (Echinochloa crus-gali and Setaria vividis) were inoculated in the surface layer in one pot filled with 16 cm² of dry field soil and covered with soil. The prescribed diluted solutions of the formulations of the respective active compounds obtained in the same manner as in Test Example 1 were sprayed to the soil upon the inoculation. The herbicidal effect and herbicide damage on crops of the respective compounds were investigated after 3 weeks from the treatment. Evaluations of the herbicidal effect and herbicide damage were carried out in the same manner as in Test Example 3. As the representative example, the test results of compound No. 1 and 24, and the comparative compound C-1 are shown in Table 12.

TABLE 12
Spraying to soil before sprouting

| Compound | Dosage | Triticum | Glycine | Echinochloa | Setaria |
|---|---|---|---|---|---|
| No. | (g ai/ha) | aestivum | max | crus-gali | vividis |
| 1 | 80 | 0 | 20 | 90 | 80 |
| 24 | 80 | 0 | 0 | 80 | 90 |
| C-1 | 80 | 0 | 20 | 0 | 0 |

### BIOLOGICAL TEST EXAMPLE 5

### Herbicidal Effect on Weeds in Dry Field and Herbicide Damage to Crops in Dry Field (Spraying Treatment to Stems and Leaves After Sprouting)

In a green house one seed each of crops in dry field (Triticum aestivum) and weeds (Veronica persica and Viola mandshurica) were inoculated in the surface layer in one pot filled with 16 cm² of dry field soil and covered with soil. After the sample plants were grown to second- and third-leaf stages at the greenhouse, the prescribed diluted solutions of the formulations of the respective active compounds obtained in the same manner as in Test Example 1 were sprayed over the plants from the above. The herbicidal effect and herbicide damage on crops of the respective compounds were investigated after 3 weeks from the treatment. Evaluations of the herbicidal effect and herbicide damage were carried out in the same manner as in Test Example 3. As the representative example, the test results of the compound No. 13 and the comparative compound C-1 are shown in Table 13.

TABLE 13
Spraying to stems and leaves after sprouting

| Compound | Dosage | Triticum | | Viola |
|---|---|---|---|---|
| No. | (g ai/ha) | aestivum | Veronica persica | mandshurica |
| 13 | 80 | 0 | 100 | 100 |
| C-1 | 80 | 0 | 60 | 30 |

### FORMULATION EXAMPLE 1

### Granules

To a mixture of the compound No. 3 of the invention (10 parts), bentonite (montmorillonite) (30 parts), talc (58 parts), and lignosulfonate (2 parts) was added water (25 parts) and the mixture was kneaded well and granulated into 10-40 mesh size by an extrusion type granulator and dried at 40 to 50° C. to obtain granules.

### FORMULATION EXAMPLE 2

### Granules

Clay mineral particles (95 parts) having a particle size distribution in a range of 0.2 to 2 mm were put in a rotary mixer and under rotating condition, the compound No. 5 (5 parts) was sprayed together with a liquid diluent to wet the particles homogeneously and then the resulting mixture was dried at 40 to 50° C. and granulated to obtain granules.

### FORMULATION EXAMPLE 3

### Emulsifiable Concentrate

The compound No. 13 (30 parts) of the invention, xylene (55 parts), polyoxyethylene alkyl phenyl ether (8 parts), and calcium alkylbenzenesulfonate (7 part) were mixed and stirred to obtain emulsions.

### FORMULATION EXAMPLE 4

### Wettable Powders

The compound No. 1 (15 parts) of the invention, a mixture of white carbon (hydrated amorous silicon oxide fine powder) and powdered clay (1:5) (80 parts), sodium alkylbenzenesulfonate (2 part), and sodium alkylnaphthalenesulfonate-formalin condensate (3 parts) were mixed in pulverized form to obtain wettable powders.

### FORMULATION EXAMPLE 5

### Water-Dispersible Granules

The compound No. 1 (20 parts) of the invention, sodium ligninsulfonate (30 parts), bentonite (15 parts), and calcined diatomaceous earth powder (35 parts) were well mixed, water was added, extruded and dried using a 0.3 mm screen to obtain water-dispersible granules.

1. Herbicidal compositions comprising sulfonanilides of the formula (I) 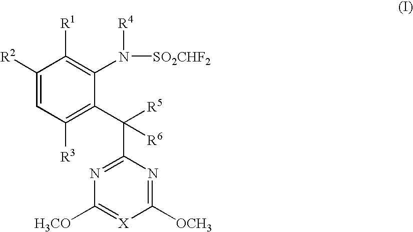
wherein
R¹ represents hydrogen, fluorine, chlorine, C₁₋₄ alkyl, C₁₋₄ alkoxy, C₃₋₆ cycloalkyl-C₁₋₄ alkyloxy or C₁₋₄ haloalkoxy,
R² represents hydrogen, fluorine or chlorine,
R³ represents hydrogen or fluorine,
R⁴ represents hydrogen or C₁₋₄ alkyl which may be optionally C₁₋₄ alkoxy-substituted, C₃₋₆ alkenyl or C₃₋₆ alkynyl,
R⁵ represents hydrogen,
R⁶ represents hydroxy, fluorine or chlorine, or
R⁵ and R⁶ may form, together with the carbon to which they are bonded, C═O, and
X represents CH or N,
provided that the following cases are excluded:
(i) R¹ represents hydrogen, fluorine or chlorine, R² represents hydrogen, R³ represents hydrogen, R⁴ represents hydrogen, R⁵ represents hydrogen, and R⁶ represents hydroxy,
(ii) R¹ represents hydrogen, fluorine or chlorine, R² represents hydrogen, R³ represents hydrogen, R⁴ represents hydrogen, and R⁵ and R⁶ form C═O together with the carbon to which they are bonded,
(iii) R¹ represents C₁₋₄ alkyl, R² represents hydrogen, R³ represents hydrogen, R⁴ represents hydrogen, R⁵ represents hydrogen, R⁶ represents hydroxy, and X represents CH, or
(iv) R¹ represents C₁₋₄ alkyl, R² represents hydrogen, R³ represents hydrogen, R⁴ represents hydrogen, R⁵ and R⁶ form C═O together with the carbon to which they are bonded, and X represents CH.

2. The herbicidal compositions set forth in claim 1, wherein
R¹ represents hydrogen, fluorine, chlorine, methyl, ethyl, methoxy, ethoxy, n-propyloxy, isopropyloxy, n-butyloxy, isobutyloxy, cyclopropylmethyloxy or difluoromethoxy,
R² represents hydrogen, fluorine or chlorine,
R³ represents hydrogen or fluorine,
R⁴ represents hydrogen, methyl, ethyl, n-propyl, n-butyl, methoxymethyl, ethoxymethyl, allyl, 2-butenyl, propargyl or 2-butynyl,
R⁵ represents hydrogen,
R⁶ represents hydroxy, fluorine or chlorine, or
R⁵ and R⁶ may form, together with the carbon to which they are bonded, C═O, and
X represents CH or N,
provided that the following cases are excluded:
(i) R¹ represents hydrogen, fluorine or chlorine, R² represents hydrogen, R³ represents hydrogen, R⁴ represents hydrogen, R⁵ represents hydrogen, and R⁶ represents hydroxy,
(ii) R¹ represents hydrogen, fluorine or chlorine, R² represents hydrogen, R³ represents hydrogen, R⁴ represents hydrogen, and R⁵ and R⁶ form C═O together with the carbon to which they are bonded,
(iii) R¹ represents methyl or ethyl, R² represents hydrogen, R³ represents hydrogen, R⁴ represents hydrogen, R⁵ represents hydrogen, R⁶ represents hydroxy, and X represents CH, or
(iv) R¹ represents methyl or ethyl, R² represents hydrogen, R³ represents hydrogen, R⁴ represents hydrogen, R⁵ and R⁶ form C═O together with the carbon to which they are bonded, and X represents CH.

3. The herbicidal compositions set forth in claim 1, wherein
R¹ represents fluorine, chlorine, methyl, ethyl or methoxy,
R² represents hydrogen or fluorine,
R³ represents hydrogen,
R⁴ represents hydrogen, methyl, ethyl, n-propyl, n-butyl, methoxymethyl, ethoxymethyl, allyl, 2-butenyl, propargyl or 2-butynyl,
R⁵ represents hydrogen,
R⁶ represents hydroxy, or
R⁵ and R⁶ may form, together with the carbon to which they are bonded, C═O, and
X represents N,
provided that the following cases are excluded:
(i) R¹ represents fluorine or chlorine, R² represents hydrogen, R³ represents hydrogen, R⁴ represents hydrogen, R⁵ represents hydrogen, and R⁶ represents hydroxy, or
(ii) R¹ represents fluorine or chlorine, R² represents hydrogen, R³ represents hydrogen, R⁴ represents hydrogen, and R⁵ and R⁶ form C═O together with the carbon to which they are bonded.

4. The herbicidal compositions set forth in claim 1, wherein
R¹ represents hydrogen, fluorine, chlorine, methyl, methoxy, ethoxy, n-propyloxy, isopropyloxy, n-butyloxy, isobutyloxy, cyclopropylmethyloxy or difluoromethoxy,
R² represents hydrogen, fluorine or chlorine,
R³ represents hydrogen or fluorine,
R⁴ represents hydrogen, methyl, ethyl, n-propyl, n-butyl, methoxymethyl, ethoxymethyl, allyl, 2-butenyl, propargyl or 2-butynyl,
R⁵ represents hydrogen,
R⁶ represents hydroxy, fluorine or chlorine, and
X represents CH,
provided that the following cases are excluded:
(i) R¹ represents hydrogen, fluorine or chlorine, R² represents hydrogen, R³ represents hydrogen, R⁴ represents hydrogen, R⁵ represents hydrogen, and R⁶ represents hydroxy, or
(ii) R¹ represents methyl, R² represents hydrogen, R³ represents hydrogen, R⁴ represents hydrogen, R⁵ represents hydrogen, R⁶ represents hydroxy, and X represents CH.

5. Sulfonanilides of the formula (IA) 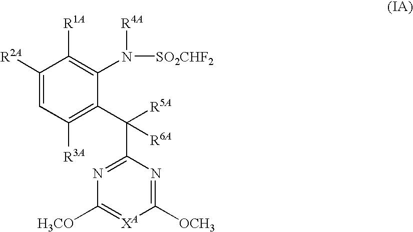
wherein
R^{1A} represents methyl, ethyl, methoxy, ethoxy, n-propyloxy, isopropyloxy, n-butyloxy, isobutyloxy, cyclopropylmethyloxy or difluoromethoxy,
R^{2A} represents hydrogen, fluorine or chlorine,
R^{3A} represents hydrogen or fluorine,
R^{4A} represents hydrogen, methyl, ethyl, n-propyl, n-butyl, methoxymethyl, ethoxymethyl, allyl, 2-butenyl, propargyl or 2-butynyl,
R^{5A} represents hydrogen,
R^{6A} represents hydroxy, or
R^{5A} and R^{6A} may form, together with the carbon to which they are bonded, C═O, and
X^{A} represents CH or N,
provided that the following cases are excluded:
(i) R^{1A} represents methyl or ethyl, R^{2A} represents hydrogen, R^{3A} represents hydrogen, R^{4A} represents hydrogen, R^{5A} represents hydrogen, R^{6A} represents hydroxy, and X^{A} represents CH,
(ii) R^{1A} represents methyl or ethyl, R^{2A} represents hydrogen, R^{3A} represents hydrogen, R^{4A} represents hydrogen, and R^{5A} and R^{6A} form C═O together with the carbon to which they are bonded, and X^{A} represents CH,
(iii) R^{1A} represents methoxy or difluoromethoxy, R^{2A} represents hydrogen, R^{3A} represents hydrogen, R^{4A} represents hydrogen, R^{5A} represents hydrogen, R^{6A} represents hydroxy, or R^{5A} and R^{6A} form, together with the carbon to which they are bonded, C═O, and X^{A} represents CH, or
(iv) R^{1A} represents methyl, R^{2A} represents fluorine, R^{3A} represents hydrogen, R^{4A} represents hydrogen, R^{5A} represents hydrogen, R^{6A} represents hydroxy, and X^{A} represents CH.

6. Sulfonanilides of the formula (IB) 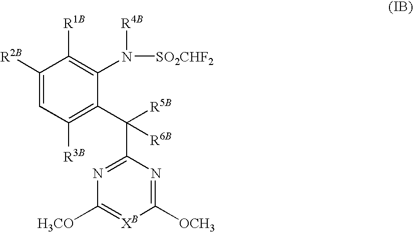
wherein
R^{1B} represents fluorine or chlorine,
R^{2B} represents hydrogen,
R^{3B} represents hydrogen,
R^{4B} represents ethyl, n-propyl, n-butyl, methoxymethyl, allyl, 2-butenyl, propargyl or 2-butynyl,
R^{5B} represents hydrogen,
R^{6B} represents hydroxy, or
R^{5B} and R^{6B} may form, together with the carbon to which they are bonded, C═O, and
X^{B} represents N.

7. Sulfonanilides of the formula (IC) 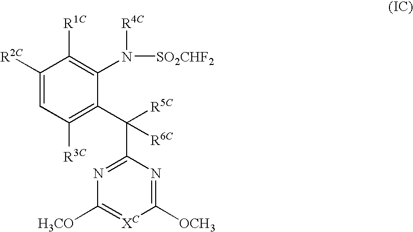
wherein
R^{1C} represents fluorine,
R^{2C} represents fluorine,
R^{3C} represents hydrogen,
R^{4C} represents hydrogen,
R^{5C} represents hydrogen,
R^{6C} represents hydroxy, fluorine or chlorine, and
X^{C} represents CH or N,
provided that
(i) where X^{C} represents N, then R^{6C} represents hydroxy, and
(ii) where X^{C} represents CH, then R^{6C} represents fluorine or chlorine.

8. A process for the preparations of the compounds of claim 5, characterized in that
(a) Preparation of the compounds of the formula (IA) wherein R^{4A} represents hydrogen and R^{5A} and R^{6A} form C═O, together with the carbon to which they are bonded:
compounds of the formula (II) 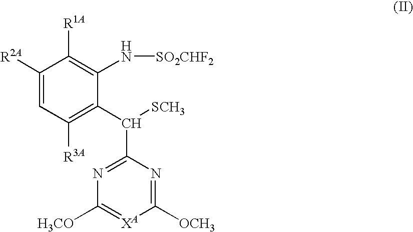
wherein R^{1A}, R^{2A}, R^{3A} and X^{A} have the same definition as aforementioned, are reacted with hydrogen peroxide and acetic acid in the presence of inert solvents, or
(b) Preparation of the compounds of the formula (IA) wherein R^{4A} represents hydrogen and R^{5A} and R^{6A} form C═O, together with the carbon to which they are bonded:
compounds of the formula (III) 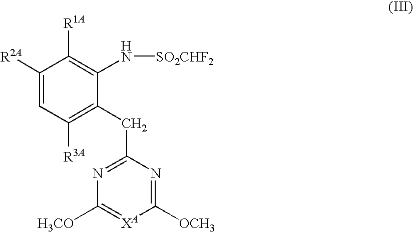
wherein R^{1A}, R^{2A}, R^{3A} and X^{A} have the same definition as aforementioned, are reacted an oxidizing agent in the presence of inert solvents, and if appropriate, in the presence of an acid catalyst,
or
(c) Preparation of the compounds of the formula (IA) wherein R^{4A} represents hydrogen, R^{5A} represents hydrogen and R^{6A} represents hydroxy:
compounds of the formula (IAc) 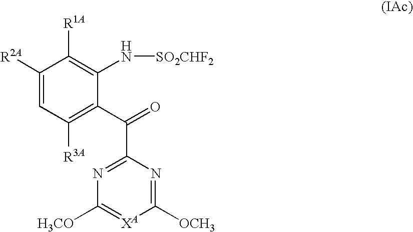
wherein R^{1A}, R^{2A}, R^{3A} and X^{A} have the same definition as aforementioned, are reacted with an alkali metal hydride complex or a borane complex, in the presence of inert solvents,
or
(d) Preparation of the compounds of the formula (IA) wherein R^{4A} represents methyl, ethyl, n-propyl, n-butyl, methoxymethyl, ethoxymethyl, allyl, 2-butenyl, propargyl or 2-butynyl:
compounds of the formula (IAd) 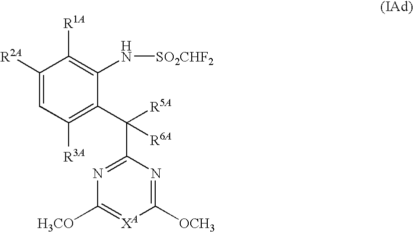
wherein R^{1A}, R^{2A}, R^{3A}, R^{5A}, R^{6A} and X^{A} have the same definition as aforementioned,
are reacted with compounds of the formula (IV)
R^{4Ad}−L^{d} (IV)
wherein R^{4Ad} represents methyl, ethyl, n-propyl, n-butyl, methoxymethyl, ethoxymethyl, allyl, 2-butenyl, propargyl or 2-butynyl, and L^{d} represents halogen, in the presence of inert solvents, and if appropriate, in the presence of an acid binder.

9. A process for the preparations of the compounds of claim 6,
characterized in that
(e)compounds of the formula (V) 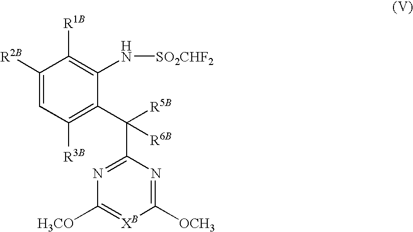
wherein R^{1B}, R^{2B}, R^{3B}, R^{5B}, R^{6B} and X^{B} have the same definition as aforementioned,
are reacted with compounds of the formula (VI)
R^{4Be}−L^{e} (VI)
wherein R^{4Be} represents ethyl, n-propyl, n-butyl, methoxymethyl, ethoxymethyl, allyl, 2-butenyl, propargyl or 2-butynyl, and L^{e} represents halogen,
in the presence of inert solvents, and if appropriate, in the presence of an acid binder.

10. A process for the preparations of the compounds of claim 7,
characterized in that
(f) Preparation of a compound of the formula (IC) wherein R^{4C} represents hydrogen, R^{5C} represents hydrogen, R^{6C} represents hydroxy and X^{C} represents N:
a compound of the formula (VII) 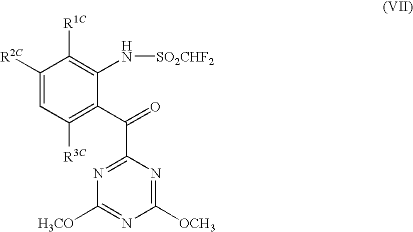
wherein R^{1C}, R^{2C} and R^{3C} have the same definition as aforementioned, are reacted with an alkali metal hydride complex or a borane complex, in the presence of inert solvents,
or
(g) Preparation of a compound of the formula (IC) wherein R^{4C} represents hydrogen, R^{5C} represents hydrogen, R^{6C} represents fluorine or chlorine and X^{C} represents CH:
a compound of the formula (ICg) 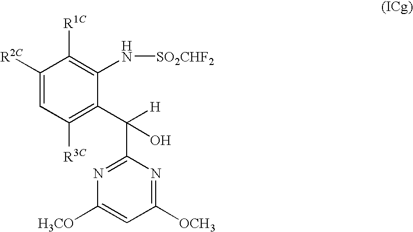
wherein R^{1C}, R^{2C} and R^{3C} have the same definition as aforementioned, are reacted with a halogenating agent, in the presence of inert solvents.

11. A process for combating weeds, characterized in that sulfonanilides of the formula (I) according to claim 1 are applied to weeds and/or their habitat.

12. A process for combating weeds, characterized in that a composition according to claim 1 is applied to weeds and/or their habitat.

13. Process for the preparation of herbicidal compositions, characterized in that sulfonanilides of the formula (I) according to claim 1 are mixed with extenders and/or surface active agents.

14. Compounds of the formula (XIV) 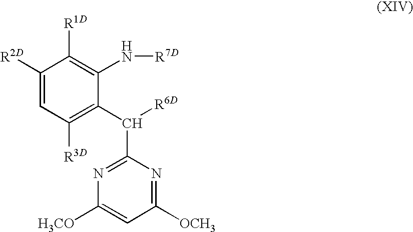
wherein
R^{1D} represents methyl, methoxy, ethoxy, n-propyloxy, isopropyloxy, n-butyloxy, isobutyloxy, cyclopropylmethyloxy or difluoromethoxy,
R^{2D} represents hydrogen, fluorine or chlorine,
R^{3D} represents hydrogen or fluorine,
R^{6D} represents hydrogen or methylthio, and
R^{7D} represents hydrogen or difluoromethanesulfonyl,
provided that the following cases are excluded:
(i) RID represents methoxy or difluoromethoxy, R^{2D} represents hydrogen, R^{3D} represents hydrogen, R^{6D} represents hydrogen or methylthio, and R^{7D} represents difluoromethanesulfonyl, or
(ii) RID represents methyl, R^{2D} represents hydrogen or fluorine, R^{3D} represents hydrogen, R^{6D} represents hydrogen, and R^{7D} represents difluoromethanesulfonyl.

15. Compounds of the formula (XV) 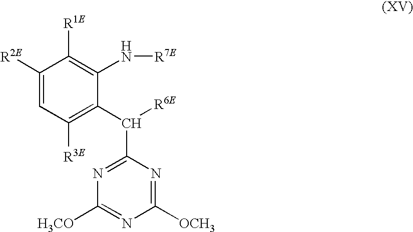
wherein
R^{1E} represents methyl, ethyl or methoxy,
R^{2E} represents hydrogen or fluorine,
R^{3E} represents hydrogen,
R^{6E} represents hydrogen or methylthio, and
R^{7E} represents hydrogen or difluoromethanesulfonyl.
